(19) United States
(12) Patent Application Publication
(10) Pub. No.: US 2010/0143319 A1
(43) Pub. Date: Jun. 10, 2010

(54) KIWIFRUIT COMPOSITIONS

(75) Inventor: Iona Elizabeth Weir, Auckland (NZ)

Correspondence Address: YOUNG & THOMPSON, 209 Madison Street, Suite 500, Alexandria, VA, 22314, US

(73) Assignee: VITAL FOOD PROCESSORS LIMITED, Auckland (NZ)

(21) Appl. No.: 12/598,733

(22) PCT Filed: May 2, 2008
(86) PCT No.: PCT/NZ2008/000098

§ 371 Date: Nov. 23, 2009

(30) Foreign Application Priority Data
May 3, 2007 (NZ) 554991
May 3, 2007 (NZ) 554992
Jul. 6, 2007 (NZ) 556395
Aug. 2, 2007 (NZ) 560323
Aug. 2, 2007 (NZ) 560324

(51) Int. Cl. A61K 38/43; A61P 1/00

(52) U.S. Cl. 424/94.1

(57) ABSTRACT

Extracts derived from kiwifruit are particularly useful for managing gut health and for treating or preventing digestive dysfunction and/or gastrointestinal tract disorders. In one aspect the extracts include an effective amount of prebiotic material derived from kiwifruit of the species Actinidia deliciosa (green kiwifruit). In another aspect the extracts include an effective amount of fibre, an effective amount of at least one enzyme and an effective amount of prebiotic material, all being derived from kiwifruit. These components work together to provide significant benefits to gastrointestinal health.

### FIELD OF INVENTION

The invention relates to kiwifruit compositions particularly for use in managing gut health and for the treatment or prevention of a variety of gastrointestinal tract disorders, and methods of manufacturing same.

### BACKGROUND OF INVENTION

Digestion is the whole complex biological process by which food is converted to fuel. The more efficient the digestion process the more energy the body derives from a given quantity of food. Many people suffer from digestive dysfunction or gastrointestinal tract disorders such as indigestion, gastric reflux, bloat, gas, abdominal pain, diarrhoea, heart-burn, constipation, irritable bowel syndrome, inflammatory bowel disease, ulcerative colitis, crohn's disease, haemorrhoids (piles), diverticular disease and cancer of the colon or large bowel.

It is known that fibre is essential for healthy bowel function. When fibre passes through the bowel it absorbs a lot of water, so it increases the bulk of the waste matter. This also makes the waste softer and increases the speed and ease with which it passes through the bowel. A diet rich in fibre has many health benefits, as it reduces the risk of a number of bowel problems.

Enzymes also play a role in the digestion process. In the human gastrointestinal system, proteolytic enzymes operate by chopping the long chain complex amino acids that make up proteins found in food into shorter chains or individual amino acids which can then pass into the cells lining the small intestine. These more simple compounds may be used as building blocks for growth and maintenance or be further processed into fuel to provide the body with energy. Some vitamins present in foods are only made available to be absorbed by the gastrointestinal lining once the material surrounding them is broken down. For example, much of the vitamin B12 in red meat would be unavailable if the proteinaceous matrix was not first hydrolysed. A fit healthy body produces most but not all the enzymes it requires for efficient digestion. However, many not so fit and healthy bodies do not produce enough enzymes for efficient digestion. The balance is derived from the food we eat. However, to prevent the body taking in too much additional enzyme by way of the food supply, enzyme inhibitors, such as enzyme suppressors in saliva and low pH in the stomach operate to control the activity of the enzymes to ensure that the material entering the stomach will not cause an enzyme overload. Accordingly, it may be difficult for the body to obtain the full balance of enzymes required for efficient digestion.

The gut microflora is also an important factor in digestive function. In humans, gut microflora comprises more than 500 different species of bacteria that have a great metabolic impact upon human health. The gut microflora can be divided into potentially deleterious and potentially health-promoting species. For example, some Clostridium species and proteolytic Bacteriodes species are considered potentially harmful because of their association with certain acute and chronic gastrointestinal complaints. Their metabolic end products are toxic and can cause cellular destruction in the bowel. On the other hand, Bifidobacterium species and the lactic acid bacteria, particularly Lactobacillus species, are considered to play an important role in a healthy gut ecosystem through their antagonistic activities towards potential pathogens, immunomodulatory activities, production of short chain fatty acids and reduction of microflora associated enzyme activities involved in the production of carcinogens and gentoxins.

There is currently much interest in the concept of actively managing the colonic microflora with the aim of improving host health. This has been traditionally attempted by the consumption of live microbial food supplements known as probiotics. Examples of commercially used probiotics are lactobacilli and bifidobacteria which are present in foods such as yoghurt.

An alternative approach is the consumption of other types of food ingredients known as prebiotics. A prebiotic is a non-digestible food ingredient that beneficially affects the host by selectively promoting the growth and/or activity of one or more health promoting bacteria in the gut, thus improving host health. For a food ingredient to be classified as a prebiotic, it should: 1) be neither hydrolysed nor absorbed in the upper part of the gastrointestinal tract; 2) be a selective substrate for one or a limited number of potentially beneficial commensal bacteria in the colon, thus stimulating the bacteria to grow or become metabolically activated or both; and 3) be able as a consequence to alter the colonic microflora toward a more healthy composition. As such, they fortify indigenous gut flora components that are thought to be of benefit.

A number of pharmaceutical or nutritional compositions for treating or preventing digestive dysfunction or gastrointestinal tract disorders are known. Examples include fibre supplements such as METAMUCIL which contain a high amount of dietary fibre and can be taken on a daily basis to aid the digestion process. However, fibre alone is not sufficient for the complete functioning of the digestive system. Other compositions containing probiotics or prebiotics or a combination thereof are also available. Known prebiotics include dietary soluble fibres such as inulin and lactulose, which are able to survive the digestion process and selectively stimulate the beneficial members of the gut microflora such as bifidobacteria, in the colon.

Some of these compositions may have unwanted side-effects such as excessive gas production, uncomfortable bloating, or may not be tolerated by the recipient. Others are based on synthetic materials or compounds, and as such may conflict with other medications. Natural or organic products are becoming increasingly popular with consumers.

Accordingly there is a need for more effective compositions for managing gut health and/or for treating or preventing digestive dysfunction and/or gastrointestinal tract disorders.

### OBJECT OF THE INVENTION

It is an object of the invention to provide extracts derived from kiwifruit that are useful for managing gut health and/or for treating or preventing digestive dysfunction and/or gastrointestinal tract disorders and/or a method of manufacturing same, which ameliorates some of the disadvantages and limitations of the known art or which at least provides the public with a useful choice.

### SUMMARY OF INVENTION

In a first aspect the invention resides in a powdered kiwifruit extract including an effective amount of prebiotic material derived from fruit of the species Actinidia deliciosa.

Preferably, the prebiotic material includes at least one compound selected from the group comprising oligosaccharides, polysaccharides, disaccharides, monosaccharides, cellulose, lignin and pectins. More preferably, the prebiotic material includes at least one compound selected from the group comprising fructo-oligosaccharides and gluco-oligosaccharides.

Preferably, the total amount of prebiotic material present in the extract is in the range of about 10-35% w/w.

Preferably, the amount of fructo-oligosaccharides present in the extract is in the range of about 5-10% w/w.

Preferably, the amount of gluco-oligosaccharides present in the extract is in the range of about 4-10% w/w.

Preferably, the prebiotic material further includes chlorophyll.

The form of the prebiotic material included in the extract is not limiting. It may be fresh or in any other form such as processed, heated or otherwise enzymatically treated.

The extract may include enzymes. The enzyme component may include enzymes extracted from kiwifruit or an enzyme complex including enzymes extracted from kiwifruit. Preferably the enzymes are derived from kiwifruit of the genus Actinidia. Kiwifruit enzymes include actinidin which is a thiol cysteine protease obtained from the fruit of Actinidia species such as Actinidia chinensis, or Actinidia deliciosa. Kiwifruit is also thought to include possibly four other protease enzymes in minor quantities and a lipase enzyme also in a minor quantity.

Preferably the extract has a proteolytic enzyme activity in the range of 100-400 U/mg. More preferably, the extract has a proteolytic enzyme activity in the range of 170-250 U/mg.

The extract may include fibre. Preferably the fibre is derived from kiwifruit of the genus Actinidia.

Preferably the fibre is present in the range of about 5-15% w/w of the extract. More preferably, the fibre is present in the range of about 6-10% w/w of the extract.

In a second aspect the invention resides in a powdered kiwifruit extract, including an effective amount of prebiotic material derived from fruit of the species Actinidia deliciosa, an effective amount of fibre, and an effective amount of at least one enzyme.

Preferably, the prebiotic material includes at least one compound selected from the group comprising oligosaccharides, polysaccharides, disaccharides, monosaccharides, cellulose, lignin and pectins. More preferably, the prebiotic material includes at least one compound selected from the group comprising fructo-oligosaccharides and gluco-oligosaccharides.

Preferably, the total amount of prebiotic material present in the extract is in the range of about 10-35% w/w.

Preferably, the amount of fructo-oligosaccharides present in the extract is in the range of about 5-10% w/w.

Preferably, the amount of gluco-oligosaccharides present in the extract is in the range of about 4-10% w/w.

Preferably, the prebiotic material further includes chlorophyll.

The form of the prebiotic material included in the extract is not limiting. It may be fresh or in any other form such as processed, heated or otherwise enzymatically treated.

The enzyme component may include enzymes extracted from kiwifruit or an enzyme complex including enzymes extracted from kiwifruit. Preferably, the enzyme(s) are derived from kiwifruit of the genus Actinidia. Kiwifruit enzymes include actinidin which is a thiol cysteine protease obtained from the fruit of Actinidia species such as Actinidia chinensis, or Actinidia deliciosa. Kiwifruit is also thought to include possibly four other protease enzymes in minor quantities and a lipase enzyme also in a minor quantity.

Preferably the extract has a proteolytic enzyme activity in the range of 100-400 U/mg. More preferably, the extract has a proteolytic enzyme activity in the range of 170-250 U/mg.

Preferably, the fibre is derived from kiwifruit of the genus Actinidia. Preferably the fibre is present in the range of about 5-15% w/w of the extract. More preferably the fibre is present in the range of about 6-10% w/w of the extract.

The powdered extract of the above aspects of the invention may be formed into compositions of known dosage forms such as tablets or capsules, or may be mixed into liquid form, or included in foodstuffs and beverages. The amount of prebiotic material, fibre, and enzyme activity present in the extract may well differ according to the particular dosage form.

In a further aspect the invention resides in the use of an extract according to the invention in the manufacture of a pharmaceutical or nutritional composition for the treatment or prevention of digestive dysfunction and/or gastrointestinal tract disorders.

In a further aspect the invention resides in the use of an extract according to the invention in the manufacture of a foodstuff or beverage for maintaining or improving the gastrointestinal health of an animal.

In a further aspect the invention resides in the use of an extract according to the invention in the manufacture of a pharmaceutical or nutritional composition for maintaining and/or restoring the intestinal flora and/or altering the gut bacterial population towards a healthier composition.

In further aspect the invention resides in the use of an extract according to the invention in the manufacture of a pharmaceutical or nutritional composition for stimulating the growth of at least one beneficial bacteria in the gut and/or inhibiting or suppressing the growth of at least one harmful bacteria in the gut.

Preferably the beneficial bacteria are selected from the group comprising probiotic bacteria. More preferably the beneficial bacteria are selected from the group comprising bifidobacteria and lactobacilli. Some examples include Lactobacillus reuteri, Lactobacillus acidophilus, Pediococcus acidilactici, and Lactobacillus plantarum.

Preferably the harmful bacteria are selected from the group comprising pathogenic bacteria. More preferably the harmful bacteria are selected from the group comprising bacteroides, clostridia, coliforms, and sulphate reducing bacteria. Some examples include Escherichia coli, Salmonella typhimurium, and Staphylococcus aureus. Most preferably the harmful bacteria are selected from the group comprising gram negative pathogenic bacteria.

In a further aspect the invention resides in the use of an extract according to the invention in the manufacture of a pharmaceutical or nutritional composition for the prevention or treatment of infection by pathogenic gut bacteria.

Preferably the pathogenic gut bacteria are selected from the group comprising bacteroides, clostridia, coliforms, and sulphate reducing bacteria. Some examples include Escherichia coli, Salmonella typhimurium, and Staphylococcus aureus.

In a further aspect the invention resides in a method of maintaining or improving the gastrointestinal health of an animal, the method comprising administering to the animal an extract or composition according to any one of the aspects of the invention described herein.

In still a further aspect the invention resides in a method of manufacturing a powdered kiwifruit extract including an effective amount of prebiotic material derived from fruit of the species Actinidia deliciosa, said method comprising the steps of
1) selecting kiwifruit with the required degree of ripeness;
2) processing the selected kiwifruit to produce a juice or pulp at a temperature of between −40° C. to 40° C. to ensure that the prebiotic material present in the kiwifruit remains intact;
3) freezing the juice or pulp within 48 hours after processing;
4) freeze drying the frozen juice or pulp and processing the dried product as desired.

In still a further aspect the invention resides in a method of manufacturing a powdered kiwifruit extract including an effective amount of prebiotic material derived from fruit of the species Actinidia deliciosa, an effective amount of fibre, and an effective amount of at least one enzyme, said method comprising the steps of:
5) selecting kiwifruit with the required degree of ripeness;
6) processing the selected kiwifruit to produce a juice or pulp at a temperature of between −40° C. to 40° C. to ensure that the enzymes and prebiotic material present in the kiwifruit remain intact;
7) freezing the juice or pulp within 48 hours after processing;
8) freeze drying the frozen juice or pulp and processing the dried product as desired.

Further aspects and advantages of the present invention will become apparent from the ensuing description which is given by way of example only.

As used herein the term “prebiotic material” refers to any non-digestible components of the kiwifruit extract that beneficially affect the host by selectively promoting the growth and/or activity of one or more health promoting bacteria in the gut.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

### DESCRIPTION OF DRAWINGS

Preferred embodiments of the invention will now be described by way of example only, with reference to the accompanying drawings in which:

FIG. 1 is a graph showing the effects of the kiwifruit extracts of the present invention on the growth of Lactobacillus plantarum;

FIG. 2 is a graph showing the effects of the kiwifruit extracts of the present invention on the growth of Pediococcus acidilactici;

FIG. 3 is a graph showing the effects of the kiwifruit extracts of the present invention on the growth of Lactobacillus acidophilus;

FIG. 4 is a graph showing the effects of the kiwifruit extracts of the present invention on the growth of Lactobacillus reuteri;

FIG. 5 is a graph showing the effects of the kiwifruit extracts of the present invention on the growth of E. coli O157:H7;

FIG. 6 is a graph showing the effects of the kiwifruit extracts of the present invention on the growth of Salmonella typhimurium;

FIG. 7 is a graph showing the effects of the kiwifruit extracts of the present invention on the growth of Staphylococcus aureus;

FIG. 8 is a graph showing the weekly defecation frequency of subjects in the first human clinical trial;

FIG. 9 is a graph showing the changes in constipation score of subjects in the first human clinical trial;

FIG. 10 is a graph showing the faecal score of subjects in the first human clinical trial;

FIG. 11 is a graph showing the change in weekly faecal frequency of subjects in the second human clinical trial;

FIG. 12 is a graph showing the mean faecal scores achieved in subjects in the second human clinical trial;

FIG. 13 is a graph showing the daily changes in the faecal score of subjects in the second human clinical trial;

FIG. 14 is a graph showing the weekly mean scores for constipation sensation of subjects in the second human clinical trial.

### DETAILED DESCRIPTION

The following description will describe the invention in relation to preferred embodiments of the invention. The invention is in no way, limited to these preferred embodiments as they are purely to exemplify the invention only and possible variations and modifications that would be readily apparent are intended to be included within the scope of the invention.

Kiwifruit is the most nutrient-rich of the top 26 fruits consumed in the world today. It also has the highest density of any fruit for vitamin C and magnesium, a limited mineral in the food supply of most affluent countries and a nutrient important for cardiovascular health. Among the top three low-sodium, high-potassium fruits, kiwifruit ranks number one, having more potassium than a banana or citrus fruits. Kiwifruit is also an excellent source of dietary fibre. Kiwifruit is also a good source of vitamin E and potassium. Kiwifruit is low fat and contains no cholesterol.

Unlike other fruits, kiwifruit has an unusually broad complement of nutrients. Most fruits tend to be high in only one or two nutrients, but kiwifruit delivers 8% DV of folic acid, 8% DV of copper, 8% DV of pantothenic acid, 6% DV of calcium and magnesium, 4% DV of iron and vitamin B6, 2% DV of phosphorus and trace amounts of vitamin A (beta carotene) and other vitamins and minerals. Kiwifruit is ranked as having the fourth highest natural antioxidant potential, next to the red fruits containing high levels of beta carotene. Kiwifruit is also particularly high in two amino acids: arginine and glutamate.

The present invention broadly relates to a powdered kiwifruit extract including an effective amount of prebiotic material derived from fruit of the species Actinidia deliciosa. The extract is useful either in powdered form or in any other suitable dosage form for the overall maintenance or improvement of gut health and/or for treating or preventing digestive dysfunction and/or gastrointestinal tract disorders. It is important that the species of green kiwifruit (that is, Actinidia deliciosa) is used to manufacture the extracts of the present invention because yellow or gold species of kiwifruit have different chemical constituents and do not contain the effective prebiotic material.

The present inventor has found that material extracted from kiwifruit using the process described herein has a prebiotic effect. The prebiotic material present in the kiwifruit extract influences the pattern of the gut microflora, with a preferential promotion of the growth of beneficial bacteria and concomitant reduction in the growth of harmful bacteria.

The extract of the present invention may also comprise fibre and enzymes, preferably also derived from kiwifruit of the genus Actinidia, but not necessarily the deliciosa species. The present inventor has found that material extracted from kiwifruit using the process described herein not only contains prebiotic material, but also contains enzymes and fibre. These components of the composition are present in sufficient amounts to provide additional benefits in respect of the health of the digestive system and the treatment and prevention of digestive dysfunction and/or gastrointestinal tract disorders.

An embodiment of the invention will now be described in which the composition is derived from kiwifruit of the species Actinidia deliciosa. The present inventor has found that if kiwifruit is processed using a particular method, a preferred composition can be obtained, which comprises at least prebiotic material, but may also include fibre, and enzyme(s) in the desired ratios and with the desired efficacy for maintaining or improving overall gut health and/or for treating or preventing digestive dysfunction and/or gastro-intestinal tract disorders. The commercial processing method has been carefully designed such that any damage or effects on the prebiotic material or the enzymes present in the kiwifruit is minimised or prevented. Therefore an extract can be obtained which retains the natural enzyme activity and prebiotic activity of the kiwifruit in sufficient concentrations to be effective.

The method used to manufacture the extract utilises some of the “soft pulping” technology referred to in New Zealand Patent No. 235972 (which is hereby incorporated by reference) to produce a pulpy green kiwifruit juice.

Firstly, the kiwifruit may undergo a pre-treatment process which may include the well known steps of ripening, inspecting, grading, and/or sorting of the kiwifruit. With regard to ripening, it is important to use ripe or mature kiwifruit when producing the compositions of the invention. This is because of two factors. Firstly, the enzyme activity of the kiwifruit peaks just prior to the fruit becoming overly ripe or ‘mushy’. Secondly, the inventor believes that the presence of the prebiotic material is linked to programmed cell death (fruit ripening). Therefore, by controlling the degree of ripeness before pulping it is possible to control the amount of enzyme and the amount of prebiotic material present in the resulting composition. Preferably the fruit is processed when it is just on the cusp of being ripe or is at the height of its ripeness, as at that stage enzyme activity will be peaking and the prebiotic material will be present in the desired ratio.

Both enzyme activity and ripeness can be measured prior to picking or processing the kiwifruit. Ripeness is measured using the Brix system. Kiwifruit with a sugar level of 12±4° Brix is ideal and indicative of ripeness. Kiwifruit exceeding this Brix level may be acceptable but are likely to be overly mature or fermenting and may not produce a composition with the required efficacy of ingredients. Kiwifruit with a Brix level below the ideal, may be artificially ripened before use. Time left in storage may be sufficient—kiwifruit picked at 5° Brix rises to 10.5° Brix in 4-6 weeks in cool storage at 0° C. This fruit will ripen to reach 12° Brix or higher upon removal from cool storage. Other changes in chemistry also occur as the kiwifruit ripens so that mature kiwifruit within the ideal range of ripeness provide a superior product.

Upon completion of the pre-treatment process, if conducted, the external surface of the kiwifruit is then sterilized. This preferably involves firstly dehairing the kiwifruit, using a thermal process to remove the hairs. More specifically, the thermal dehairing process may include an oven where a pyrolytic flaming step may be used to singe the hair of the kiwifruit. Preferably, during the flame process, the temperature of the kiwifruit does not become elevated during the burning of the hair. Short burn times and pre-cooling of the kiwifruit can result in the flesh of the kiwifruit, other than that immediately adjacent the skin, not rising above 30° C.

Once the hair of the kiwifruit is removed by the thermal dehairing process, the dehaired kiwifruit are then further sterilized. The fruit may be passed through an assembly having one or more roller brushes for removing any adhering foreign matter including singed hairs from the kiwifruit. Conventional washing techniques may then be employed, one example being the use of a series of spray nozzles. Wash additives aiding cleansing or reducing the bacteria count on the kiwifruit may be employed according to local regulations and requirements. For example, the fruit may be washed by a chlorine wash and/or an ozone impregnated water wash followed by a fresh water rinse.

The sterilized kiwifruit are then conveyed into a hopper, which is generally tapered to form a funnel directing the kiwifruit individually, that is, one by one, into a further conveyor system which conveys the fruit to a cutting assembly. The cutting assembly includes a cutting device such as a water laser or similar which has the advantage of preventing damage to the seed so the seed of the fruit does not contaminate the pulp. However, it should be apparent to those skilled in, the art that other cutting devices are suitable for use including rotating circular blades, reciprocating blades, fluid jet cutting devices, swing blades, etc.

Preferably, the cutting device cuts the kiwifruit substantially in half, preferably across its length as this has been found to result in reduced seed damage. Alternately, the cutting device may be replaced with a soft crushing device able to break the skin of the kiwifruit without causing significant seed or cellular damage to the kiwifruit. For instance, the kiwifruit may be directed between rollers to result in the breakage of the skin of the kiwifruit. For instance, the kiwifruit may be burst by passing the kiwifruit through spaced rollers biased towards each other. This squashes the fruit so the skin is split, the burst kiwifruit substantially intact but readily separable into large fragments. Other bursting methods may be employed.

After the kiwifruit are cut the semi-spherical shaped kiwifruit segments are then passed through a pressing assembly designed to separate the skin from the pulp and seed without damaging the seed coat. Generally described, the pressing assembly is adapted to perform “soft-pulping” operations. The term “soft-pulping” relates to a pulping or comminution process which is relatively mild and gentle compared to many conventional fruit pulping techniques. Soft-pulping is characterised by only a minor proportion (generally less than 5-10%) of seeds being fragmented. Further, there is no significant disintegration or lysis of fruit cells or components. Excluded from the meaning of soft-pulping processes are chemical and/or enzyme lysis methods, thermal techniques, techniques directed to the breaking down of cells, and mechanical techniques which involve excessive pulverisation of fruit material.

In a preferred embodiment, the pressing assembly performs the “soft-pulping” of the kiwifruit by pressing the kiwifruit segments between a twin converging belt press. The press belts may be endless loops rotated about a series of pulleys. The distance separating the press belts preferably decreases in the direction of travel of the kiwifruit. This results in increasing pressing forces being exerted upon the kiwifruit as the kiwifruit travel along the length of the pressing assembly. This action results in the soft pulping of the kiwifruit without significant damage to the seeds so the seeds do not contaminate the pulp.

The pulp generated from the pressing assembly is then directed to a screening process, in order to separate the seeds from the pulp. Generally, the pulp is separated from the seed using a soft mechanical screening technique. This preferably involves the use of a pulp finisher including a rotating flexible impeller which is rotated within a cone shaped screen having apertures of a predetermined size. The size of the apertures is preferably selected to permit the pulp and juice of the kiwifruit to pass through the screen while retaining a substantial portion, if not all, of the seeds within the interior cavity defined by the screen.

The resulting separated pulp is then put through a freeze concentrating step. It is important that the pulp is frozen as soon as possible after it is produced as, this provides for a “fresher” and more superior product. Little, if any, of the beneficial properties of the composition are lost if the freezing process is carried out quickly (that is, within about 48 hours), and the pulp can be stored frozen for a long period of time without any adverse effects. Freeze concentrating methodology is well known and need not be described in any further detail herein. However, the inventor has found that blast freezing is particularly effective. The pulp is generally frozen in standard sized pales which are used to collect the fresh pulp after processing. The pulp can be stored frozen until it is required to make the composition.

When it is required, the frozen pulp is then freeze dried. Freeze drying methodology is well known and need not be described in any further detail herein. The freeze drying cycle is generally about 36 hours. The longer the freeze drying cycle, the better the resulting product as the enzymatic and prebiotic activity is retained. The resulting dried product may then be milled into a powder which can then be utilised as appropriate, for example it may be encapsulated, tableted or added to or incorporated in other products.

Preferably, the resulting dried product or powder is encapsulated, and each capsule contains approximately 535 mg of the dried product or powder. The capsule preferably also contains other excipients. Preferred excipients include isomalt, magnesium stearate, and silicon dioxide.

Throughout the whole process, it is important to ensure that the temperature remains below that which causes significant degradation of the enzymes and the prebiotic material present in the kiwifruit. It is important that the entire process is performed at a temperature of less than 40° C. Preferably the entire process is performed at a temperature of between −40° C. to 40° C., and even more preferably, the entire process is performed at a temperature of between −10° C. to 10° C. Preferably, this temperature range is adhered to during the entire process, including the storage of the whole fruit, prior to it being broken open and/or “soft pulped”. In any case, it is essential to ensure that once the fruit has been broken open that the temperature ranges indicated above are utilised throughout the entire process.

By controlling the temperature of the process it ensures that the enzymes and prebiotic material present in the kiwifruit is not degraded and remains intact to produce a composition with the desired efficacy and/or ratio of components. In addition, the cold temperatures avoid oxidation taking place so no reducing agents are required, so the resulting product can be “organically” certified.

Kiwifruit extracts obtained by way of the processing technique described above have been analysed to determine their chemical composition. By way of example only, the tables below show the results of this analysis in respect of three different batches of extract. The first example is an analysis of a batch of extract in pure powdered form. Examples 2 and 3 show an analysis of two different batches of extract in capsule form.

### Example 1

### Kiwifruit Extract (Powder Form)

| Constituent | Amount |
|---|---|
| Energy kilojoules KJ/100 g | 1560 |
| Protein g/100 g | 4.1 |
| Fat g/100 g | 3.1 |
| Saturated Fat g/100 g | 0.5 |
| Moisture (Vacuum Oven) g/100 g | 2.3 |
| Carbohydrate (By Difference) g/100 g | 76.5 |
| Sugars g/100 g | 63.6 |
| Fibre (dietary) g/100 g | 9.8 |
| Sodium mg/100 g | 30 |
| Ash g/100 g | 4.2 |

### Example 2

### Kiwifruit Composition (Capsule Form)

| Constituent | Amount |
|---|---|
| Energy kilojoules KJ/100 g | 1530 |
| Protein g/100 g | 4.4 |
| Fat g/100 g | 3.6 |
| Saturated Fat g/100 g | 0.6 |
| Moisture (Vacuum Oven) g/100 g | 4.3 |
| Carbohydrate (By Difference) g/100 g | 74.0 |
| Sugars g/100 g | 50.5 |
| Fibre (dietary) g/100 g | 8.5 |
| Sodium mg/100 g | 24 |
| Ash g/100 g | 5.2 |

### Example 3

### Kiwifruit Composition (Capsule Form)

| Constituent | Amount |
|---|---|
| Energy kilojoules KJ/100 g | 1590 |
| Protein g/100 g | 3.4 |
| Fat g/100 g | 3.5 |
| Saturated Fat g/100 g | 0.6 |
| Moisture (Vacuum Oven) g/100 g | 2.8 |
| Carbohydrate (By Difference) g/100 g | 79.4 |
| Sugars g/100 g | 55.5 |
| Fibre (dietary) g/100 g | 6.2 |
| Sodium mg/100 g | 36 |
| Ash g/100 g | 4.7 |

The above examples show that the chemical constituents can vary slightly between extracts. The dietary fibre component of the extract has been further analysed and found to include cellulose, lignin and pectins. The total amount of dietary fibre present in the extracts generally ranges between about 5-15% w/w. The total amount of carbohydrate present in the extracts generally ranges between about 70-90% w/w. The carbohydrate component includes sugars such as sucrose, glucose, fructose, maltose, lactose, galactose and starch. The amount of each of these components may vary from batch to batch.

The carbohydrate component also includes fructo-oligosaccharides and gluco-oligosaccharides. Of the total amount of carbohydrate present in the extracts, it is believed that approximately 5-20% is prebiotic material. The prebiotic material present in the extracts includes one or more of the following compounds: oligosaccharides, polysaccharides, disaccharides, monosaccharides (from the carbohydrate component of the extract), cellulose, lignin and pectins (from the fibre component of the extract). Thus the prebiotic effect of the extract likely comes from a combination of the prebiotic material present in the carbohydrate and fibre components of the extract. It is likely that the prebiotic effect is a result of the combined activity of the various forms of prebiotic material present in the extracts. However, it is believed that the majority of the prebiotic effect is caused by the fructo-oligosaccharides and gluco-oligosaccharides present in the extracts.

Combining the amount of prebiotic material from both the carbohydrate and fibre components of the extract, the total amount of prebiotic material present in the extract is in the range of about 10-35% w/w.

The amount of fructo-oligosaccharides present in the extract is in the range of about 5-10% w/w, and the amount of gluco-oligosaccharides present in the extract is in the range of about 4-10% w/w.

The extracts may also include chlorophyll.

The present inventor has therefore surprisingly found that the kiwifruit extracts of the present invention contain prebiotic material which helps promote growth of beneficial bacteria in the gut to obtain a healthy balance of gut microflora. The inventor believes that the prebiotic material also suppresses the growth of harmful bacteria.

By using the technique described herein to extract the prebiotic material from kiwifruit, an effective pharmaceutical or nutritional composition can be formulated for the purpose of balancing microflora in the gut to promote a healthy digestive system.

The extracts of the present invention may also include enzymes. The enzyme component may include enzymes extracted from kiwifruit or an enzyme complex including enzymes extracted from kiwifruit. Preferably the enzymes are derived from kiwifruit of the genus Actinidia. Kiwifruit enzymes include actinidin which is a thiol cysteine protease obtained from the fruit of Actinidia species such as Actinidia chinensis, or Actinidia deliciosa. Kiwifruit is also thought to include possibly four other protease enzymes in minor quantities and a lipase enzyme also in a minor quantity.

Several batches of the kiwifruit extract of the present invention were analysed to determine the proteolytic enzyme activity of the extracts. The assay used to determine the proteolytic activity of the extracts involves the reaction of the kiwifruit extract with the substrate azocasein using suitable buffers and a dye, then measuring the liberated azocasein dye with a spectrophotometer at 420 nm and comparing it to a zero time control. Enzyme activity is determined as units/mg (U/mg) where 1 enzyme unit equals an increase of 0.0005 absorbance units at 420 nm per hour at 35° C. at pH 6.25. The following method was used:

1. Prepare the kiwifruit extract solution (25 mg/ml) using both water and 50 mM citrate buffer containing 10 mM EDTA (2 ml each for every 100 mg powdered extract).
2. Similarly prepare the substrate (azocasein) solution (5 mg/ml) using both water and 50 mM citrate buffer containing 10 mM EDTA (10 ml each for every 100 mg substrate).
3. Add 0.25 ml of kiwifruit extract solution into 0.5 ml substrate solution. Note down the time of reaction.
4. Make a zero control sample by adding 0.5 ml of 8% Tri Chloro Acetic Acid (TCA) into the kiwifruit extract solution and then adding the substrate.
5. Incubate the original sample at 35° C. for at least 2 hours.
6. After incubation, add 0.5 ml 8% TCA to stop the reaction. Centrifuge the tubes to remove any un-reacted substrate.
7. From the supernatant, pipette out 0.75 ml into another tube. Neutralise it by adding 50 μL of 2N Sodium Hydroxide.
8. Measure the solution using the spectrophotometer at 420 nm. Record the absorbance and convert into enzyme units.

A number of batches of the extract were tested for enzyme activity, and some selected examples are set out in the following table:

| | Enzyme activity |
|---|---|
| Batch Number | (U/mg) |
| 0236401 | 141.76 |
| 0249602 | 119.7 |
| 0250402 | 126.4 |
| 0410401 | 168.0 |
| 0404604 | 148.48 |
| 955421 | 148.4 |
| 960046 | 159.8 |
| 960203 | 164.8 |
| 963214 | 174.4 |
| 991409 | 154.56 |
| 711140 | 150.28 |
| 0551105 | 185.92 |
| 0619304 | 205.68 |
| 0650201 | 191.04 |
| 0713404 | 188.4 |
| 0334501 | 153.92 |
| 0340202 | 192.96 |
| 0410404 | 180.48 |
| 0419504 | 197.4 |
| 0545302 | 209.6 |
| 0545502 | 224.5 |
| 0549504 | 173.44 |
| 0550604 | 168.32 |
| 0551105 | 185.92 |
| 0619304 | 205.68 |
| 0632701 | 176.32 |
| 0647405 | 191.01 |
| 0650201 | 191.04 |
| 0713404 | 188.4 |
| 0730405 | 177.6 |
| 0419505 | 310.12 |
| 0425301 | 290.33 |
| 0433205 | 276.16 |
| 0445405 | 270.08 |

From the various tests, the present inventor has found that an effective range of proteolytic enzyme activity in the extracts of the invention is between 100-400 U/mg, with the preferred range for efficacy being between 170-250 U/mg. If the enzyme activity is less than 100 U/mg, the extract will not be effective at stimulating motility in the gut. If the enzyme activity is higher than 400 U/mg, this will have a negative effect on gut health.

The inventor believes that the particular combination of fibre, enzymes and prebiotic material present in the extracts provides a unique benefit for the entire gastrointestinal tract resulting in improved digestive function.

When difficult and irregular bowel movements are experienced they are often associated with an imbalance in the gut microflora where the bad bacteria present in the gut outweigh the good bacteria. The compositions of the present invention initiate a series of events which work in unison to promote regularity and the right balance of gut microflora so that the digestion system works the way it is supposed to.

The enzymes in the extracts of the present invention work in two ways. Firstly, they aid motility (or movement) of waste product gently through the digestive system. This occurs after the initial dose of the composition if there is a solid or difficult stool to move. The enzymes affect receptors in the gut which tell the muscles to contract and keep moving waste product gently through the system. The enzymes are very similar to those produced by the digestive system itself so once the initial solid stool has been passed, they help to digest food so that subsequent stools move more freely through the digestive tract. This enzyme digestion also aids with absorbing the optimum level of nutrients into the system from the food consumed.

Once the initial solid stool is passed via the motility effect of the enzymes, the fibre in the extracts of the present invention works by providing bulk to subsequent stools. The stools bulk increases by absorbing moisture from the system into the stool which helps it move through the digestive tract more comfortably.

It is believed that the prebiotic material present in the extracts of the invention aid in long term bowel health. The main role of the prebiotic material is to provide food for the good bacteria in the digestive tract to grow. This process has a dual effect. Firstly, it helps restore the balance of gut microflora and helps protect the digestive wall so it can work normally. Secondly, if there is a low level of good bacteria in the system it means that the system is not producing enough enzymes on its own to digest food properly. Encouraging the growth of the good bacteria encourages the growth of the enzymes so that the body can continue to digest food the way it is supposed to. It is the prebiotic material that will support the long term, proper functioning of the digestive system.

When these three key ingredients are introduced into the digestive system, they effectively work together to promote a healthy digestive function, both in the short and long term. The present inventor believes that the three ingredients act synergistically to obtain superior benefits to digestive function than that obtained when each of the ingredients acts individually. There is clearly a working inter-relationship between the components of the composition which provides significant advantages.

The powdered extracts of the present invention may be incorporated into different dosage forms and thus the amounts of fibre, prebiotic material and level of enzyme activity may well differ according to the particular dosage form.

The extracts are preferably incorporated into tablets or capsules. However the extracts may be made into liquid compositions, or included in foodstuffs and beverages. For example, the compositions of the present invention may be incorporated in frozen or chilled desserts; blended with sugar or prepared as a sprinkle on product for use on breakfast cereals and fruit; incorporated into a wide variety of drinks and beverages; blended with milk or cream; blended with yoghurt, or ice cream; encapsulated and administered orally or as a suppository; pressed into tablet form to be administered orally; formed into a drench for oral administration.

Dosage forms such as capsules and tablets may comprise further additives as known in the art, for example, tableting aids, flavouring agents, texture improving agents, product improving additives, and the like. Such tableting aids and additives are well known and utilised for many vitamin and mineral preparations (for example Vitamin C capsules or tablets), and need not be described in any further detail here. Any amount or ratios of these optional additives may be utilised as required or as desired or as it is determined by the intended use of the composition or by health regulations. Examples of suitable additives or excipients include isomalt, magnesium stearate and silicon dioxide.

An advantage of the extracts being administered orally by way of capsules or tablets is that the enzymes contained in the extracts are not denatured by enzyme suppressors in the saliva, so that when the capsule or tablet is swallowed it passes into the stomach and allows the enzymes to be released into the stomach where they begin working immediately.

It has been found that a dosage rate of one or two capsules or tablets (each containing about 535 mg of the powdered extract) daily before food is preferred in order to maintain or provide long term digestive health benefits. However, to obtain short term benefits from the composition, for example, for immediate relief of constipation, the dosage rate may be increased, for example, up to about four capsules daily (two before the morning meal and two before the evening meal)° depending on the severity of the constipation, for as long as required to relieve the symptoms. Preferably the composition is administered approximately 15-45 minutes prior to meals.

It is envisaged that the extracts of the present invention will be useful for managing, maintaining and/or improving overall digestive health. They may also be particularly suitable for addressing digestive dysfunctions that suggest a malfunction of the mammalian gastrointestinal system, such as indigestion, gastric reflux, bloat, gas, abdominal pain, diarrhoea, heart-burn, constipation, irritable bowel syndrome, inflammatory bowel disease, ulcerative colitis, crohn's disease, haemorrhoids (piles), diverticular disease and cancer of the colon or large bowel. However, it is to be understood and appreciated that the invention is not to be limited to such use.

It is envisaged that the extracts of the invention could be suitable for both human and animal use.

### Efficacy Studies

### Prebiotic Effects

In order to test the efficacy of the extracts of the present invention as a prebiotic, kiwifruit was processed in accordance with the processing technique as described herein to obtain an extract. Two forms of the extract were used in the tests, the first in capsule form containing excipients, and the second in pure powder form.

Tests were conducted in vitro to investigate the effect of this kiwifruit extract on the growth of the probiotic bacteria Lactobacillus reuteri, Lactobacillus acidophilus, Pediococcus acidilactici, and Lactobacillus plantarum. Tests were also conducted in vitro to investigate the effect of the kiwifruit extract on the growth of the pathogenic bacteria E. coli O157:H7, Salmonella typhimurium, and Staphylococcus aureus.

Four commonly used probiotics were selected for the studies based on their role in gastrointestinal health. Lactobacillus reuteri is indigenous to the gut, and helps to strengthen the body's natural defenses against harmful bacteria, and maintain equilibrium in the gastrointestinal tract by repopulating the non pathogenic microflora in the gut. L. reuteri secretes reuterin, a substance with antimicrobial properties that helps suppress the growth of pathogenic microorganisms in the gut. Pediococcus acidilactici are well known probiotic strains and improve gastrointestinal health by accelerating the rates of lactic acid production and decreasing the gastrointestinal pH. Lactobacillus acidophilus strains readily adapt to the human gut. Research has demonstrated that successful implantation of L. acidophilus in the human gut has many health benefits including immuno-modulation, symptomatic relief in mucous colitis, irritable colon, idiopathic ulcerative colitis, and various disorders complicated with constipation and biliary symptoms. Lactobacillus plantarum colonizes from the saliva of the mouth through to the gut mucosa, and is commonly found in fermented plant material. Research has demonstrated that L. plantarum by its fermentation activity reduces abdominal bloating associated with irritable bowel syndrome (IBS). L. plantarum is also important in immune-modulation of the gut mucosa and in particular in reducing mucosal inflammation. Thus, L. plantarum plays a major role in managing IBS. Furthermore, L. plantarum has been shown to increase IL-10 expression (anti-inflammatory cytokine) in the intestinal mucosa and therefore is of benefit to IBS, but also increases IL-10 in the islet of the pancreas thereby improving diabetes.

Three common food-borne pathogenic bacterial strains were also selected for the studies. These were E. coli O157:H7, Salmonella typhimurium, and Staphylococcus aureus. These three bacterial strains are all enteric pathogenic bacteria, in which E. coli O157:H7 and Salmonella typhimurium are gram-negative, and Staphylococcus aureus is gram-positive.

### Materials and Methods

### Probiotic Bacterial Strains

Lactobacillus reuteri, Lactobacillus acidophilus, Pediococcus acidilactici, and Lactobacillus plantarum sourced from Bioactives Research New Zealand (BRNZ) culture collection were used in this study as representative bacterial strains.

### Reagents

MRS broth, yeast extract, bacteriological peptone, lactose, and trypticase (Oxiod, England). L-cysteine hydrochloride, K₂HPO₄, KH₂PO₄, NaHCO₃, NaCl, CaCl₂, and MgSO₄ (Sigma, USA).

### Kiwifruit Extracts

Two forms of kiwifruit extract were tested. The first extract (referred to herein as “Extract 1”) was made into a composition in capsule form and contained the excipients isomalt, magnesium stearate and silica dioxide. The second extract (referred to herein as “Extract 2”) was in pure powder form. These extracts were obtained using the kiwifruit processing technique as described herein. The extracts were combined with ethanol or water at a concentration of 1 g per 20 mL. This solution was then gently rotated at either 36° C. or 45° C. for 24 hours. After 24 hours, the solution was then centrifuged at 8000 rpm for 10 minutes. The supernatant was filtered through a 0.45 μm sterile filter and then stored in a freezer for further use.

### Placebo

An isomalt placebo was provided in encapsulated granule form. 10% (w/v) ethanol extracts were prepared by taking 1.0 g of the isomalt powder and dissolving in 10 ml of Milli-Q water and gently rotating and incubating at either 36° C. or 45° C. for 24 hours.

### Bacterial Culture

The bacteria were incubated with the kiwifruit extracts in PYL broth and the optical density (OD) value was measured using a Genova UV/Vis Spectrophotometer S$-002 Jenway, UK. Bacteria were cultured in MRSC broth at 37° C. for 20 hours. A 0.1 ml aliquot of the culture was transferred into 5 ml of PYL broth (pH 6.0±0.1) containing kiwifruit extracts (final concentration 0.5% (w/v)), and incubated at 37° C. for 20 hours. PYL broth without bacteria was used as a blank for zeroing the OD value. PYL broth without kiwifruit extract was used as the control. Since the kiwifruit extract is slightly turbid, the background turbidity was determined by subtracting the sample OD from the control OD. After incubation, each of the broth were mixed well prior to measuring the OD (610 nm) value which was indicative of the growth of the bacteria. The experiment was carried out in triplicate, twice.

### Pathogenic Bacteria

Common food-borne pathogen strains used included E. coli O157:H7 (strain 2988), Salmonella typhimurium (ATCC 1772), and Staphylococcus aureus (ATCC 2592), which were obtained from Bioactives Research New Zealand. Pathogens were: first recovered from −80° C., with Brain Heart Infusion (BHI, Oxoid, England) broth. A loop full of each thawed pathogen was transferred aseptically to 5 ml of BHI in respectively marked 20 ml bottles, and then incubated at 37° C. for 20 h. After 20 h incubation, pathogen growth was checked using a haemocytometer via light microscopy to confirm log-phase state. A 0.1 ml aliquot of 1/4 diluted pathogen culture was transferred into 4 ml of BHI broth (pH 7.4±0.2) containing 0.1 ml of each kiwifruit extract (final concentration 0.25% (w/v), and incubated at 37° C. for 20 h. BHI broth with 0.1 ml 1/4 diluted pathogens only was used as negative control. No positive control was assigned. Pure BHI broth was used as a blank for equilibrating the spectrophotometer (manufacturer) to zero optical density (OD) value. After incubation, each extracted sample was mixed well prior to reading on the spectrophotometer, with OD values measured at wavelength 610 nm at room temperature. The OD values collected from the spectrophotometer at 610 nm, are indicative of pathogen growth (the higher the optical density, greater the number of living bacteria). All controls and samples had to be diluted 2 times with Mili-Q water in order to remove the turbidity and obtain zero for blanks. The test was carried out in triplicate, with the results analyzed using t-test.

### Results

### 1. Efficacy of Kiwifruit Compositions in Stimulating Growth of Probiotic Bacteria In Vitro

Effects of the Kiwifruit Compositions on the Growth of Lactobacillus plantarum

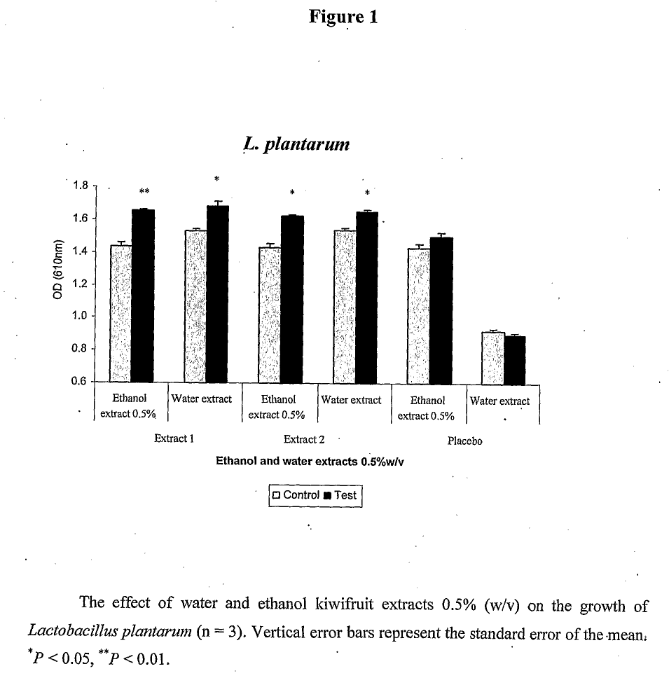

FIG. 1 shows that broth containing the kiwifruit extracts with either water or ethanol had significantly higher OD values than the controls containing no kiwifruit extract and the placebo (isomalt) control. These results demonstrate that the kiwifruit compositions promoted the growth of Lactobacillus plantarum.

Effects of the Kiwifruit Compositions on the Growth of Pediococcus acidilactici

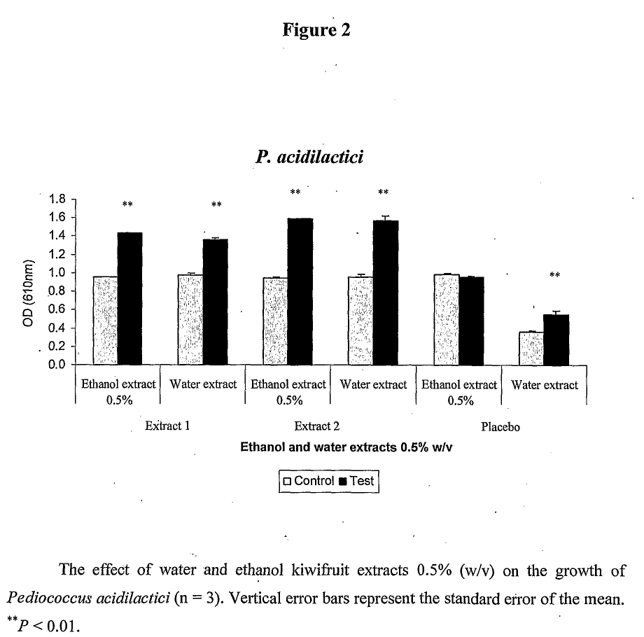

FIG. 2 shows that broth containing the kiwifruit extracts with either water or ethanol had a significantly higher OD values than the controls containing no kiwifruit extract and the placebo (isomalt) control. These results show that the kiwifruit compositions promoted the growth of Pediococcus acidilactici.

Effects of the Kiwifruit Compositions on the Growth of Lactobacillus acidophilus

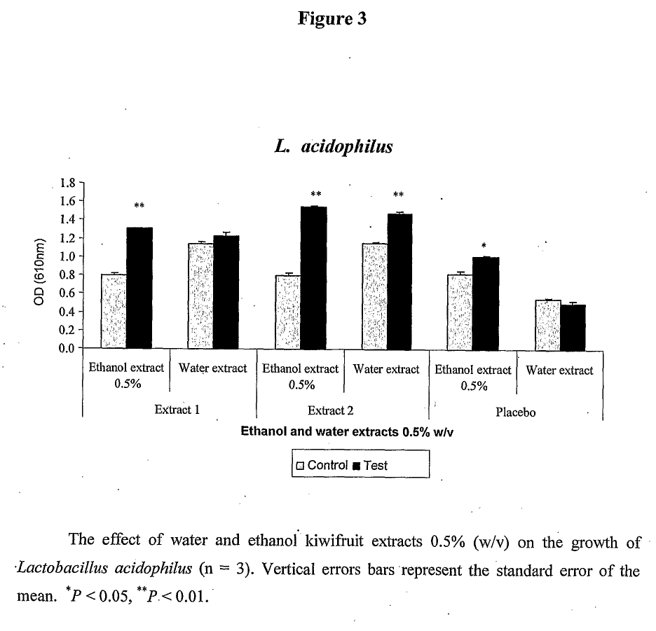

FIG. 3 shows that broth containing the kiwifruit extracts with either water or ethanol had significantly higher OD values than the controls containing no kiwifruit extract and the placebo (isomalt) control. These results demonstrate that the kiwifruit compositions promoted the growth of Lactobacillus acidophilus.

Effects of the Kiwifruit Compositions on the Growth of Lactobacillus reuteri

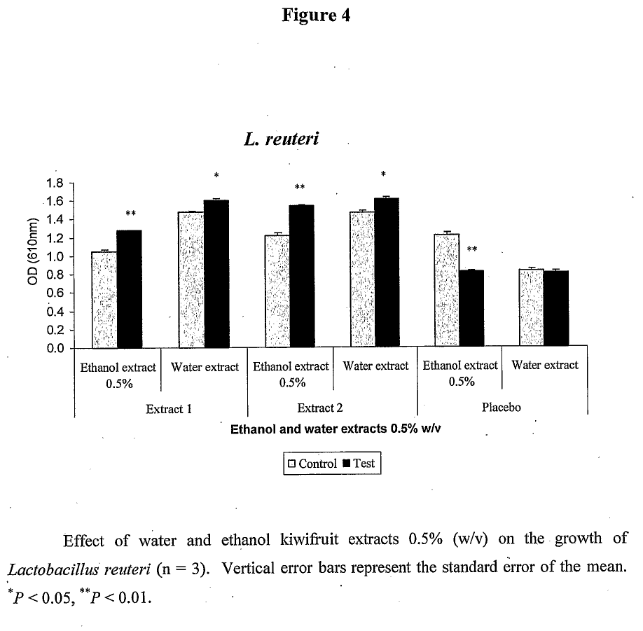

FIG. 4 shows that broth containing the kiwifruit extracts with either water or ethanol had significantly higher OD values than the controls containing no kiwifruit extract and the placebo (isomalt) control. These results show that the kiwifruit compositions promoted the growth of Lactobacillus reuteri. The ethanol placebo (isomalt) control had a significant inhibitory effect on the growth of Lactobacillus reuteri.

### 2. Efficacy of Kiwifruit Compositions in Inhibiting Growth of Pathogenic Bacteria In Vitro

In order to analyse whether the kiwifruit compositions would promote or inhibit the growth of pathogenic bacteria, kiwifruit extracts combined with water and an isomalt placebo were tested against four pathogenic bacteria: E. coli O157:H7, Salmonella typhimurium and Staphylococcus aureus.

Effects of the kiwifruit compositions on the growth of E. coli O157:H7

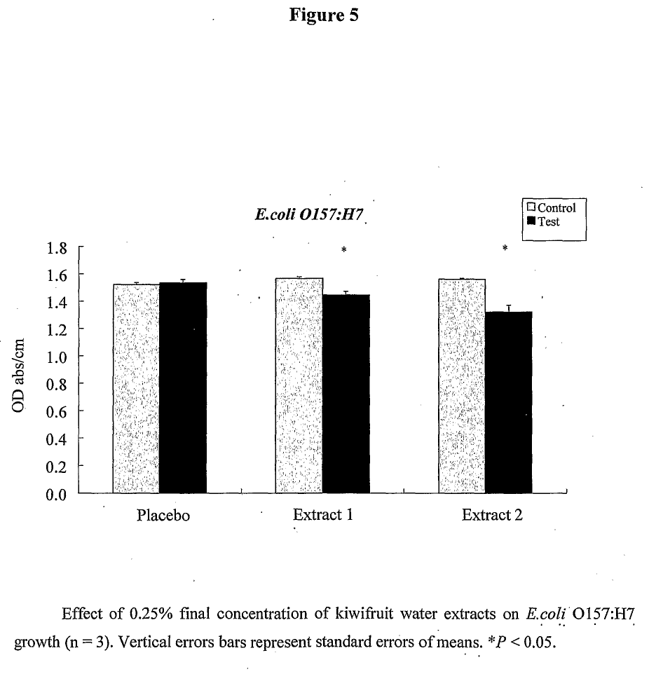

FIG. 5 shows that the kiwifruit extracts have significantly (P<0.05) lower OD values than the controls, while there was no obvious difference between the placebo and the blank controls. These results show that the kiwifruit compositions had an inhibitory effect on the growth of E. coli O157:H7. Extract 2 (the powdered form) had a greater effect than Extract 1 (the capsule form).

Effects of the Kiwifruit Compositions on the Growth of Salmonella typhimurium

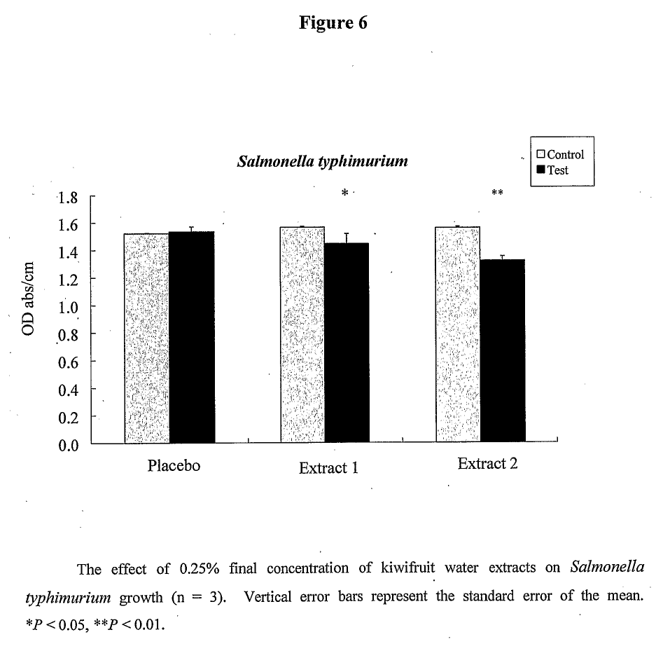

FIG. 6 shows that the kiwifruit extracts have significantly (P<0.05) lower OD values and thus bacterial growth than the controls, while there was no obvious difference between the placebo and the blank controls. These results show that the kiwifruit compositions had a significant inhibitory effect on the growth of Salmonella typhimurium. Extract 2 (the powdered form) had a greater effect than Extract 1 (the capsule form).

Effects of the Kiwifruit Compositions on the Growth of Staphylococcus aureus

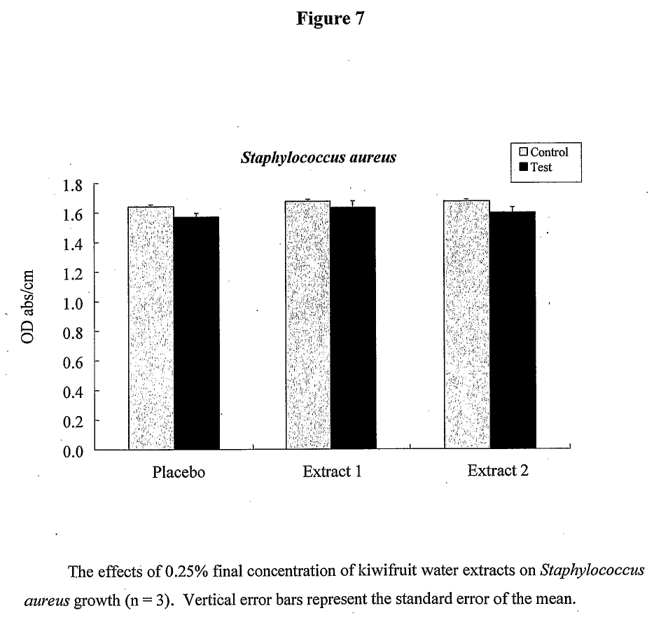

FIG. 7 shows that since the standard error of the mean is slightly high, no significant effect was found with either of the extracts on the growth of Staphylococcus aureus. However, the graph shows slight inhibition of Staphylococcus aureus growth compared to the controls.

### Deactivation of Enzyme

The enzymes (namely the protease complex) within the kiwifruit extract are heat sensitive and are deactivated at 38° C. To test whether or not the enzymes have any effect on stimulating the probiotic bacteria, the enzyme complex was deactivated by heating to 45° C. For the tests at both 36° C. and 45° C. there was no difference in the growth of probiotic or pathogenic bacteria and thus the enzymes present in the kiwifruit extract do not appear to play a role in the prebiotic activity of the kiwifruit extracts.

### Discussion

The above results demonstrate that the kiwifruit compositions do significantly stimulate the growth of the four probiotic bacteria tested, and furthermore that these results are statistically significant over the placebo control used, that is, isomalt.

The inventor has found that the kiwifruit extracts of the invention' have a “prebiotic” component which helps to regulate the gut microflora. This “prebiotic” component or “probiotic bacterial growth stimulant” is believed to be a result of one or more of the following compounds present in the kiwifruit extracts: oligosaccharides, polysaccharides, disaccharides, monosaccharides, cellulose, lignin and pectins. It is likely that the prebiotic effect is a result of the combined activity of the various forms of prebiotic material present in the extracts. However, it is believed that the majority of the prebiotic effect is caused by fructo-oligosaccharides and/or gluco-oligosaccharides present in the extracts.

The results show that there is some variation in response between the pure powdered kiwifruit extract and the capsule form in the tests in respect of all four probiotic bacteria. This variation directly correlates to the difference in concentration between the two dosage forms. The placebo contains isomalt which is a potent food source for bacteria and thus it was expected that there would be some growth of the bacteria in the presence of the placebo.

The effects of the kiwifruit extracts on the growth of the probiotic bacteria were found to be statistically significant in comparison to the placebo which demonstrates that the promotion of probiotic bacterial growth was more than just as a food source but a “true stimulant” effect. The results show that the kiwifruit extracts also inhibited the growth of the gram negative pathogenic bacteria E. coli and Salmonella typhimurium, with some inhibition of Staphylococcus aureus. This suggests that within the kiwifruit extracts there is more than just a food source, but rather some bioactive component which stimulates probiotic bacterial growth whilst inhibiting some pathogenic bacteria, particularly gram negative pathogenic bacteria.

### Effects on Digestive Health

Further tests were carried out to determine the effects of the kiwifruit extracts on digestive health. Firstly, to ascertain the mode of action and dose response curve for the compositions, two animal trials were carried out, which were then used as a basis for two double-blinded placebo-based clinical trials assessing the efficacy of the kiwifruit extracts on the relief of constipation.

The kiwifruit compositions used in these trials were in capsule form containing freeze-dried kiwifruit extract powder (360 mg) and excipients (magnesium stearate, isomalt and silicon dioxide) at 0.44 g/capsule. The recommended dosage for humans is 2 capsules twice a day based on an average adult body weight of 60 kg, that is, 1.76 g/60 kgBW, equivalent to 0.029 g/kgBW.

### Animal Efficacy Trials

The first animal trial, for supporting the human clinical trial at 4 capsules per day, tested doses equivalent to 2, 4 and 12 capsules per day. These values were based on what had been described as most effective for the composition, with the higher dose testing for the presence of diarrhoea as a result of taking the composition. The second animal trial for supporting the human trial at 6 capsules per day, tested doses equivalent to 3, 6 and 18 capsules per day.

### Criteria Established for Ascertaining Efficacy of the Tested Product for Constipation Relief

In the hypothesis model of bowel movement, if the mice in the experimental subgroups have significantly higher ink progression ratio in the small intestine than the positive controls, then the results are positive, and vice versa.

In the hypothesis model of constipation, if the mice in the experimental subgroups have a significantly shorter time of excreting the first melena faecal stool (contains ink) than the positive controls; and either the experimental mice have a significantly higher number of faecal stools within 5 hours than the positive controls; or the experimental mice have a significantly higher faecal weight than the positive controls, then the results are positive, and vice versa.

Therefor, if either the timing of the first black (melena) faecal stool or the ink progression ratio is positive, and either one of the 5 hour parameters for number of faecal stools or the 5 hours parameter for weight of the faeces is positive the results indicate that the experimental sample is efficacious for relieving constipation in mice.

### First Animal Trial

### Materials and Methods

Experimental animals: 100 Kunmin male mice (class two) were purchased from the animal center in the Academy of Military Medical Sciences of the Chinese PLA, with a weight of 16 to 18 g.
Group 1: 50 mice were randomly divided into 5 experimental groups each of 10 mice (negative control group, model control group and three dosage groups), to test small intestine motility.
Group 2: 50 mice were randomly divided into 5 experimental groups each of 10 mice (negative control group, model control group and three dosage groups), to measure the following parameters: time of first melena defecation, the number of faeces pellets, the weight of faeces, and to observe the shape of the faeces. Number of qualitative qualification is SCXK M 2002-001.

Dosage Calculation: the recommended adult dose (based on an adult body weight of 60 kg) is 2 capsules twice daily. Each capsule is 0.44 g thus the total daily dose is 1.76 g, equal to a dosage of 0.029 g/kg BW for an adult weighing 60 kg.

For this experiment, the dosage was set at 10 times that of the recommended dosage, namely 0.29 g/kgBW per day (4 capsules). The experiment was designed with a low dosage group (0.145 g/kgBW) (2 capsules) and a high dosage group (0.87 g/kgBW) (12 capsules). The kiwifruit extract was dissolved by gentle agitation into sterile distilled water. For both the negative control group (0 g/kgBW) and the model control group sterile distilled water was used as the treatment. Oral intubation was used to administer the dose to the mice, at a measured quantity of 20 ml/kgBW.

Instruments and reagents used: surgical scissors, forceps, ruler, injector, balance, active carbon powder, Arabic gum, Diphenoxylate Tablets (contain 2.5 mg Diphenoxylate per tablet).

### Methodology

### Reagent Preparation

Arabic gum ink solution preparation: 100 g Arabic gum was added to 800 ml of water, and gently heated until boiling to clarify the solution. Subsequently, 50 g of active carbon (powder), was added to the solution, and gently brought to boiling three times. The solution was cooled and then distilled water was added to bring the final volume to 1000 ml. The solution was stored in the fridge at 4° C., and gently shaken prior to use.

Diphenoxylate suspension (0.50 g/l) preparation: twenty tablets of Diphenoxylate (containing 2.5 mg Diphenoxylate per tablet), were triturated in a mortar to powder, and water added to a final volume of 100 ml just prior to use.

Diphenoxylate suspension (0.25 g/l) preparation: ten tablets of Diphenoxylate (containing 2.5 mg Diphenoxylate per tablet), were triturated in a mortar to powder, and water added to a final volume of 100 ml just prior to use.

### Small Intestine Motility Experiment

8 days after oral administration of the different treatments, each group was fasted for 16 hours (with free access to drinking water). Mice in the Peristaltic Inhibition Model control group and the three dose groups were orally administered 0.25 g/l, diphenoxylate at a measured quantity of 20 ml/kg BW. The negative control group was administered distilled water. 30 minutes later, the 0.145 g/kgBW, 0.29 g/kg BW and 0.87 g/kg BW dosage groups were orally administered the ink suspension containing the corresponding dosage. The 0 g/kg BW group (negative control group and model control group) were orally administered blank inks with a measured quantity of 20 ml/kg BW.

Twenty five minutes later, the mice were sacrificed, and the abdominal space opened to separate the mesentery. The intestines were cut off from the pylorus to the appendices. The small intestine was gently pulled into a straight line, and the small intestine total length measured, and the total length of ink progression measured i.e. is the length from the pylorus to the ink advancing frontline. Thus if the treatment improved motility then the ink would progress further due to increased peristaltic movement.

To calculate the rate of ink progression ratio:

Ink progression ratio=the length of ink progression/small intestine total length

### Constipation Experiment

Parameters measured in the animal trial were time of defecation, the number of faeces pellets, the weight of the faeces, and visual observation of the shape of the faeces

The different doses of kiwifruit composition were orally administered to the mice, and the shape of the faeces during the eight day feeding trial was observed. After 8 days of treatment with the kiwifruit composition, each group was starved for 16 hours (with free access to drinking water). The mice in the Peristaltic Inhibition Model control group and the three dose groups were orally administered 0.50 g/l of diphenoxylate in the calculated volume of 20 ml/kg BW, whilst the negative group were given an equivalent volume of water. 30 minutes later, the 0.145 g/kgBW, 0.29 g/kgBW and 0.87 g/kgBW group were orally administered the ink suspension containing the corresponding dosage. The 0 g/kgBW group (negative control group and model control group) were orally administered blank ink solutions at a volume of 20 ml/kg BW. Each mouse was then housed in a separate cage, and allowed to take food and water as usual. The time of the first melena defecation was recorded, along with the number of faecal pellets within 5 hours and the weight of the faeces, as well as the shape of the faeces.

Statistical Analysis of the Experimental Data

SPAA software was used to statistically analyze the experimental data. Either t-test (homogeneity of variance) or t° test (heterogeneity of variance) were used to compare the negative control group and the model control group. The comparison between each dosage group and the model control group was tested for homogeneity of variance. The data which met the requirement of “homogeneity of variance” were statistically analyzed using single factor analysis of variance and the mean number of multiple comparisons of several experimental groups and the control (model control). When the data met the requirement of “normal equal variance”, the data was converted and statistically analyzed.

### Results

### Criteria for Confirming Efficacy:

If either the timing of the first melena defecation or the rate of ink progression should be positive, in combination with either the number of faecal pellets or the weight of faeces also positive, then the tested substance can be determined as having a significant effect on mouse laxation.

### The Effect on Mouse Small Intestine Motility by Kiwifruit Extract

TABLE 1
The effect on mouse small intestine motility by diphenoxylate
( x̅ ± SD)

| | Number | Ink Progression | |
|---|---|---|---|
| Dose | (animal) | (%) | P value |
| 0 g/kg BW (negative control) | 10 | 72.1 ± 10.6 | 0.000 |
| 0 g/kg BW (model control) | 10 | 33.8 ± 7.0## | |

Compared with negative control group,
##P < 0.01

Diphenoxylate inhibits peristalsis in the small intestine and thus has been used in these animal experiments as a negative control. The further the ink progresses the more peristaltic motility there is in the small intestine i.e. the less constipated. The rate of ink progression in the negative group and the model control group meet the requirement of variance homogeneity and thus were analyzed by t-test. As can be seen in table 1, the rate of ink progression in the model control group has a statistically significant (P<0.01) decrease compared to the negative control group. Therefore the use of this Peristaltic Inhibition Model by the oral administration of 0.25 g/L diphenoxylate is valid.

TABLE 2
The effect of kiwifruit compositions on the rate of ink progression
( x̅ ± SD)

| | Number | Ink Progression | |
|---|---|---|---|
| Dose | (animal) | (%) | P value |
| 0 g/kg BW (model control) | 10 | 33.8 ± 7.0 | |
| 0.145 g/kg BW | 10 | 41.3 ± 9.0 | 0.070 |
| 0.29 g/kg BW | 10 | 43.2 ± 8.6* | 0.025 |
| 0.87 g/kg BW | 10 | 56.7 ± 10.8** | 0.000 |

Compared with the model control group,
*P < 0.05,
**P < 0.01

Different doses of the kiwifruit composition were orally administered to the mice for 8 days, with the ink progression in the mice significantly meeting the requirement of normal equal variance of the data and also meeting the requirement of “homogeneity of variance” when statistically analyzed by single factor analysis of variance and the method of mean number multiple comparisons of several experiment groups and a control (model control). As can be seen in table 2, the rate of ink progression in the middle and high dosage groups has a significant (P<0.05 and P<0.01 respectively) increase compared to the model control group. Therefore this shows that the kiwifruit compositions at a dosage of 0.29 g/kg BW and 0.87 g/kgBW increased the rate of ink progression when assessed using the Mouse Peristaltic Inhibition Model.

Deactivation of the enzyme complex by heating the powder to 65° C. for 1 hour resulted in identical motility rate to that of the control. This suggests therefore that the enzyme complex is affecting the motility of the small intestine.

The Effect of Kiwifruit Composition on the Time of the First Melena (Black from Ink) Defecation

TABLE 3
The effect on the time of first melena defecation by diphenoxylate
(x ± SD)

| | | Time of first | |
|---|---|---|---|
| | Number | melena defecation | |
| Dose | (animal) | (min) | P value |
| 0 g/kg BW (negative control) | 10 | 83.1 ± 51.9 | 0.000 |
| 0 g/kg BW (model control) | 10 | 231.7 ± 81.7## | |

Compared with the negative control group,
##P < 0.01.

The time of the first melena defecation in the negative control and the model control group met the requirement of homogeneity of variance, and thus the data was analyzed using t-test. As observed in table 3, the time of the first melena defecation in the model control group has very high significance (P<0.01) compared to the model control group. Hence the treatment of mice by oral dosing with 0.25 g/L diphenoxylate is valid as a Constipation Model.

TABLE 4
The effect of kiwifruit composition on the time of the first melena
defecation ( x̅ ± SD)

| | | Time of first | |
|---|---|---|---|
| | Number | melena defecation | |
| Dose | (animal) | (min) | P value |
| 0 g/kg BW (model control) | 10 | 231.7 + 81.7 | |
| 0.145 g/kg BW | 10 | 213.5 ± 58.3 | 0.521 |
| 0.29 g/kg BW | 10 | 199.3 ± 46.0 | 0.256 |
| 0.87 g/kg BW | 10 | 182.8 ± 59.9 | 0.090 |

Different doses of the kiwifruit composition were orally administered to mice for 8 days. To test the homogeneity of variance for the time of the first melena defecation, the data which met the criteria for “homogeneity of variance” were statistically analyzed by the method of single factor analysis of variance and the method of mean number multiple comparisons of several experiment, groups and a control (model control). As can be seen in table 4, comparison of the time of first melena defecation between the dosage groups and the model control groups, shows no significant statistical difference (P>0.05).

With the statistically significant result achieved in the “Small Intestine Motility Experiment” it is clear that motility is a key component in relation to the mode of action of the kiwifruit composition. That experiment eliminated the need for the digestive system to cope with digestion of any other food due to the mice being starved for 16 hours prior to treatment. The lack of any statistical significance in this time to “first melena defecation experiment” is a key indicator that a higher dosage rate is required to effectively relieve constipation when the digestive system is also required to cope with the digestion of other food from a normal diet. Motility is stimulated but not enough at the selected dosage rates to overcome the resistance of food/waste already present.

### The Effects of Kiwifruit Composition on the Number of Faeces Pellets

TABLE 5
The effect on the number of faeces pellets of diphenoxylate ( x̅ ± SD)

| | | Number of | |
|---|---|---|---|
| | | faeces pellets | |
| | Number | defecated within | |
| Dose | (animal) | 5 hours (grain) | P value |
| 0 g/kg BW (negative control) | 10 | 59 ± 15 | 0.000 |
| 0 g/kg BW (model control) | 10 | 25 ± 16 | |

Compared with negative control group,
##P < 0.01

The number of faecal pellets in the negative control and model control groups met the requirement of homogeneity of variance, and thus the data was analyzed using t-test. As can be seen in table 5, the number of faecal pellets in the model control group has a significant (P<0.01) decrease compared to the model control group. Thus, confirming the use of oral administration of 0.50 g/L diphenoxylate to mice as a valid Constipation Model.

TABLE 6
The effects of kiwifruit composition on the number of faeces pellets
( x̅ ± SD)

| | | Number of faeces | |
|---|---|---|---|
| | | pellets defecated | |
| | Number | within 5 hours | |
| Dose | (animal) | (grain) | P value |
| 0 g/kg BW (model control) | 10 | 25 ± 16 | |
| 0.145 g/kg BW | 10 | 24 ± 13 | 0.899 |
| 0.29 g/kg BW | 10 | 27 ± 11 | 0.736 |
| 0.87 g/kg BW | 10 | 38 ± 22 | 0.072 |

Different dosages of the kiwifruit composition were orally administered to mice for 8 days. The data on the number of faecal pellets in each group which met the requirement of “homogeneity of variance” was analyzed using single factor analysis of variance combined with analysis of the mean number multiple comparisons of several experiment groups and a control (model control). As can be seen in table 6, comparison of the number of faecal pellets between the different dose groups and the model control group, showed no significant statistical difference (P>0.05).

### The Effects of Kiwifruit Composition on the Weight of the Faecal Pellets

TABLE 7
The effect on the weight of faeces by diphenoxylate (x ± SD)

| | | Number of faecal | |
|---|---|---|---|
| | | pellets defecated | |
| | Number | within 5 hours | |
| Dose | (animal) | (grain) | P value |
| 0 g/kg BW (negative control) | 10 | 1.12 ± 0.31 | 0.000 |
| 0 g/kg BW (model control) | 10 | 0.47 ± 0.26 | |

Compared with the negative control group,
##P < 0.01

The weight of the faeces in the negative control and model control groups met the requirement of homogeneity of variance, and thus the data were analyzed using t-test. As can be seen in table 7, the weight of the faeces in the model control group had a significant (P<0.01) decrease compared to the model control group, therefore confirming the use of the oral administration of 0.50 g/L diphenoxylate to mice as a valid Constipation Model.

TABLE 8
The effect of kiwifruit composition on the weight of the faecal pellets
(x ± SD)

| | | Number of faecal | |
|---|---|---|---|
| | | pellets defecated | |
| | Number | within 5 hours | |
| Dose | (animal) | (grain) | P value |
| 0 g/kg BW (model control) | 10 | 0.47 ± 0.26 | |
| 0.145 g/kg BW | 10 | 0.47 ± 0.22 | 0.948 |
| 0.29 g/kg BW | 10 | 0.58 ± 0.23 | 0.410 |
| 0.87 g/kg BW | 10 | 0.74 ± 0.35 | 0.033 |

Compared with the model control group,
*P < 0.05

Different doses of kiwifruit composition were orally administered to mice for 8 days. The data on the faeces weight in each group which met the requirement of “homogeneity of variance” was analyzed using single factor analysis of variance combined with analysis of the mean number multiple comparisons of several experiment groups and a control (model control). As can be seen in table 8, comparing the weight of the faecal pellets between the 0.87 g/kgBW group and the control group, there is a significant difference (P<0.05), showing that the 0.87 g/kgBW treatment with the kiwifruit composition increased the weight of the faeces in the mouse Constipation Model.

### The Effects of Kiwifruit Composition on the Shape of the Faecal Pellets

Different doses of the kiwifruit composition were orally administered to the mice for 8 days, with the shape of the faecal pellets in the negative control group, model control group and each dosage group being oval grain, with no observed diarrhoea in any dosage group.

### Conclusion

As indicated in the animal trial, after oral dosing with the kiwifruit composition for 8 days, there is no body weight increase difference among the mice in each group. The results of the small intestine motility experiment indicate that the kiwifruit composition significantly increased the rate of ink progression in the Peristaltic Inhibition Model of mice with doses of 0.29 g/kg BW and 0.87 g/kg BW; and as shown in the defecation weight of faeces experiment, 0.87 g/kgBW the kiwifruit composition significantly improved the weight of faeces defecated within 5 hours in the Constipation Model of mice. The characteristic of the faeces in each dosage group was oval grain, with no observed diarrhoea in any of the dose groups. Therefore, according to the Standard of Judgment for Laxation in the Technical Standards for Testing & Assessment of Health Food, 2003>, the results of the animal experimentation of the kiwifruit composition for laxation is considered significantly positive. The results suggested that the dose required for severe constipation was between 4 and 8 capsules per day and that no diarrhoea occurred at the higher doses. It was apparent from these results that the enzyme effect stimulated the peristalsis of the small intestine.

### Second Animal Trial

In the second animal trial, 120 healthy male BABL/c mice weighing between 18-20 grams were selected and randomly divided into 2 groups. 60 mice in group I were further separated into 5 subgroups with 12 in each (negative control, positive model control and 3 dose examining groups) to test stimulation of bowel movements (or small intestinal peristalsis). The other 60 mice in II were also sub-grouped into negative control and positive model control and 3 dosage subgroups, with 12 in each. Group II were tested for weight, shape and number count of rats' first melena (black from ink) stool. The feeding dose for this trial was based on a recommended daily intake of 3 capsules twice a day for an adult weighing 60 kg. This is equivalent to 0.044 g/kg BW. The experimental test dose was 10 times this, i.e. 0.44 g/kgBW (6 capsules). Upper limit and lower limit groups were also designated as 0.22 g/kgBW (3 capsules) and 1.32 g/kgBW (18 capsules) respectively, equal to 5 times and 30 times of the adult recommended daily intake. Test samples were dissolved with gentle agitation into sterile distilled water. All mice were lavaged via mouth with the relative dose of samples or water only, with the lavaged volume equivalent to 20 ml/kgBW.

### Methodology

### Bowel Movement (or Intestinal Peristalsis) Test

Group I: 10 days after lavage feeding with various food samples, all mice were starved for 16 hours (water still supplied). During this period, the positive control subgroup and the 3 dosage subgroups were lavaged 20 ml/kgBW with 0.25 g/l diphenoxylate, while the negative control subgroup were lavaged with water only. After 30 minutes, the dosage sub-groups of 0.22 g/kgBW, 0.44 g/kgBW and 1.32 g/kgBW were lavaged with 20 ml/kgBW of the respective dose of kiwifruit extract in Gum Arabic ink fluid. Negative and positive control subgroups (0 g/kgBW) were fed with Gum Arabic ink fluid only. After 25 minutes, all mice were executed by cervical vertebra dislocation without pain. The abdominal cavity was then opened and the mesentery separated, and then the intestinal canal was cut from the pylorus to the ileocecus, with the small intestine pulled straight to measure the length. Ink progression was defined as the length from the pylorus to the ink front. So, the ink impelling ratio can be calculated as:

Ink Impelling Ratio=(Ink Impelling Length (cm)/small intestine length (cm))×100%

### Defecation Frequency, Number Count, Weight and Shape of Faecal Stools

Group II: Mice were lavage-fed with the respective feeding dose of kiwifruit composition. Faeces characteristics of the mice in each subgroup were observed. After 9 days of feeding, all subgroups were fasted for 16 hours (water still supplied). The constipation positive control subgroup and 3 dosage subgroups were lavage-fed 20 ml/kgBW with 0.50 g/l diphenoxylate, negative control subgroup were lavage fed with only water of the same amount. After 30 minutes, the 3 dosage subgroups (0.22/kgBW, 0.44 g/kgBW, 1.32 g/kgBW) were lavaged with 20 ml/kgBW of the respective amount of the kiwifruit extract in Gum Arabic ink fluid, while negative and positive control subgroups (0 g/kgBW) were lavaged with 20 ml/kgBW Gum Arabic ink only. Mice were kept individually in separated single cages, routinely fed and watered. Observation records included time of every rat's first melena faecal stool, number count and weight of faecal stools within 6 hours, and the shape of the faeces.

### Data Analysis

All data were analyzed using SPSS. Negative and positive control subgroups were compared using t-test (2-sample test with equal standard error and different standard errors); each test subgroup was compared with the positive control by t-test for equal variance first to find the homogeneity variance, then one-way ANOVA and paired mean t-test to analyze paired experimental subgroup and control subgroup.

### Results

### Effects of Kiwifruit Composition on Mice Body Weight

Mouse weight in each group was tested for equal variances first to find homogeneity variance, then one-way ANOVA and paired mean t-test to analyze paired experimental subgroup and control subgroup.

From table 9, groups of 0 g/kgBW (negative control), 0.22 g/kgBW, 0.44 g/kg BW and 1.32 g/kg BW were compared with the positive control for original body weight, and the result shows that there were no significant differences (P>0.05), thus the assumption is confirmed that there was no significant difference in all treatments of the mice from their original body weight.

TABLE 9
Body weight changes of each group (mean x̅ ± SD)

| | Group I (n = 12) | | Group II (n = 12) | |
|---|---|---|---|---|
| | Original | After | Original | After |
| Dose | (g) | (g) | (g) | (g) |
| 0 g/kg BW | 21.9 ± 0.7 | 31.8 ± 1.5 | 21.0 ± 0.4 | 31.5 ± 2.0 |
| (+ve control) | | | | |
| 0 g/kg BW | 21.9 ± 0.7 | 21.9 ± 0.7 | 21.0 ± 0.5 | 32.2 ± 2.1 |
| (−ve control) | | | | |
| 0.145 g/kg BW | 22.0 ± 0.6 | 31.7 ± 2.2 | 20.8 ± 0.4 | 31.9 ± 2.0 |
| 0.29 g/kg BW | 21.8 ± 0.6 | 32.9 ± 1.2 | 20.9 ± 0.4 | 32.1 ± 2.0 |
| 0.87 g/kg BW | 21.9 ± 0.7 | 32.1 ± 1.7 | 21.1 ± 0.6 | 31.4 ± 2.1 |

### Effects of Kiwifruit Composition on the Small Intestine Movement in Mice

Mice were orally fed with the kiwifruit composition for 10 days. Data were analyzed by one-factor ANOVA and t-test. Results (table 10) show that the kiwifruit composition at doses of 0.44 g/kg BW and 1.32 g/kg BW obviously increased the ink-promoting rate (P<0.05 and P<0.01, respectively).

TABLE 10
Effect of kiwifruit composition on ink-progression in mouse
small intestine □ x̅ ± SD□

| | | Ink-progression | |
|---|---|---|---|
| Dose | Animals | rate (%) | P value |
| 0 g/kg BW (model control) | 12 | 31.8 ± 8.3 | |
| 0.22 g/kg BW | 12 | 37.1 ± 6.6 | 0.112 |
| 0.44 g/kg BW | 12 | 40.7 ± 9.9* | 0.0123 |
| 1.32 g/kg BW | 12 | 53.2 ± 12.2** | 0.0002 |

Compared with model control
*P < 0.05,
**P < 0.01

### Effects of Kiwifruit Composition on the Time of Excrement of the First Faeces

Mice were orally fed with kiwifruit composition for 9 days. Data was analyzed by one-factor Anova and t-test. Results (Table 11) show significant difference between 0.44 g/kgBW test group and the model control group (P<0.001), as well as the 1.32 g/kgBW test group and the model control group (P<0.001).

TABLE 11
Effect of kiwifruit composition on time of excrement of the
first faeces in mice ( x̅ ± SD□

| | | Time of excrement | |
|---|---|---|---|
| | | of the first faeces | |
| Dose | Animals | (min) | P value |
| 0 g/kg BW (model control) | 12 | 237.9 ± 11.6 | |
| 0.22 g/kg BW | 12 | 223.5 ± 18.2 | 0.0590 |
| 0.44 g/kg BW | 12 | 211.9 ± 14.3** | 0.0007 |
| 1.32 g/kg BW | 12 | 182.3 ± 12.9** | 0.0000 |

Compared with the model positive control
**P < 0.001

### Effects of Kiwifruit Composition on the Amount of Faeces

Mice were orally fed the kiwifruit composition for 9 days. Data was analyzed by one-factor Anova and t-test. Results (table 12) show significant differences between test groups of 0.44 g/kgBW and 1.32 g/kgBW with the model control group (P<0.01).

TABLE 12
Effect of kiwifruit composition on the amount of faeces in mice
( x̅ ± SD)

| | | Amount of faeces | |
|---|---|---|---|
| Dose | Animals | with 5 h (pellets) | P value |
| 0 g/kg BW (model control) | 12 | 23 ± 5 | |
| 0.22 g/kg BW | 12 | 26 ± 4 | 0.122 |
| 0.44 g/kg BW | 12 | 35 ± 9** | 0.001 |
| 1.32 g/kg BW | 12 | 46 ± 10** | 0.000 |

Compared with negative control
**P < 0.01

### Effects of Kiwifruit Composition on the Weight of Faeces

Mice were orally fed the kiwifruit composition for 9 days. Data was analyzed by one-factor ANOVA and t-test. Results (table 13) show that the test subgroups of 0.44 g/kgBW and 1.32 g/kgBW significantly increased the faeces weight (P<0.01).

TABLE 13
Effects of kiwifruit composition on the faeces weight of mice (x ± SD)

| | | Faeces weight | |
|---|---|---|---|
| Dose | Animals | within 5 h (g) | P value |
| 0 g/kg BW (Negative control) | 12 | 0.45 ± 0.09 | |
| 0.22 g/kg BW | 12 | 0.51 ± 0.07 | 0.118 |
| 0.44 g/kg BW | 12 | 0.69 ± 0.18** | 0.001 |
| 1.32 g/kg BW | 12 | 0.89 ± 0.21** | 0.000 |

Compared with the model control
**P < 0.01

### Effects of Kiwifruit Composition on the Faeces Properties

During the 9-day medication, no abnormal properties of faeces were found in any group.

### Summary

Testing the efficacy of the kiwifruit composition in the Peristaltic Inhibition Model:
The rate of ink progression in the test group was significantly higher than in the model control group, and thus it can be determined the index result is positive.
The timing of the first melena defecation in the test group is significantly less than in the model control group, and so it can be determined the result is positive.
The number of faecal pellets in 5 hours is significantly higher than in the model control group, and thus the result is positive.
The weight of the faeces defecated within 5 hours is significantly higher than in the model control group, and hence the result is positive.
No abnormal properties of faeces were found in any of the dose groups.
Administering the kiwifruit composition for 9-10 days did not have a negative effect on the body weight increase.
Administering the kiwifruit composition for 9-10 days increased the efficacy as can be seen by increased significance. For example in the first trial 12 capsules was required to achieve significance (P,0.03) for weight of faecal pellets, but with longer dosing, only 6 capsules is required (P<0.001). Therefore, the prebiotic effect takes time to build up in the system.
No diarrhoea was ever observed, even at the equivalent of 18 capsules per day.
The results of the animal trials strongly suggest that 6 capsules/day is the requirement for serious constipation.
Apart from bowel motility, for efficacy in the other parameters of constipation such as timing, weight and number of pellets, long-term feeding (greater than 6 days) is required.

### Human Efficacy Trials

Further tests were conducted in relation to the gut motility, stool formation and IBS symptoms of patients with moderate to severe constipation in two prospective double-blinded placebo controlled clinical trials. The hypothesis being tested was that the kiwifruit compositions exert a constipation relief effect on various components of the gastrointestinal tract, possibly through alterations in gut motility, bulking of the stool and equilibration of the gut micro-flora. The primary aim of the tests was to quantify any changes in frequency of bowel movements in response to the kiwifruit compositions. The secondary aim was to assess the effects of the kiwifruit compositions on symptoms of patients with moderate to severe constipation and correlate the clinical outcome with changes in faecal score and IBS symptoms.

These two trials were designed to compare the efficacy of the kiwifruit compositions in relation to dosage, length of treatment and severity of condition. The first trial was carried out at Kaixian Traditional Chinese Medicine Hospital with 60 patients suffering from moderate to severe constipation. The second trial was completed at Tianjing Centre Hospital, which is part of the China CDC (Centre for Disease Control) network through Beijing Medical University with 134 patients suffering from moderate to severe constipation. This facility is Chinese FDA registered with CNAL (Chinese National Accreditation Laboratories) certification and membership of ILAC (International Laboratory Accreditation Committee).

The findings of the first animal trial indicated that statistical significance would be difficult to achieve in the clinical trial at the rate of four capsules per day. This was because the clinical trial was designed to treat severe constipation, with the results from this animal trial suggesting that severe constipation would require a higher dose. Combined with this, the in vitro research on the prebiotic efficacy of the kiwifruit compositions provided strong evidence that the kiwifruit compositions had a three-way mode of action: namely enzyme, prebiotic and fibre. Also of utmost importance was the result that no diarrhoea had been observed, as would normally be associated with high doses of a motility stimulant. Thus it was safe to increase the dose.

Taking the animal trial results and the in vitro results together, it was apparent that the enzymes present in the kiwifruit extract stimulated the peristalsis of the small intestine. However the enzyme effect at the tested dosage levels was not strong enough to resolve serious constipation. What was now clear was that the fibre and prebiotic effects of the kiwifruit compositions provided the long-term efficacy and relief of the other parameters of constipation and accordingly, the design of the human clinical trials would need to take this into account.

In order for the clinical trials to see the effects of the prebiotic a follow up period was built into the end of each trial. This was due to the strong indication that the prebiotic effect would linger after taking the kiwifruit composition and would produce a significant difference between the placebo and treatment group.

### First Clinical Trial

### Clinical Trial Protocol:

Kiwifruit composition: a light green powder extracted from kiwifruit using the processing technique described herein, in capsule form, was used in the trials.
Dosage: Four capsules per day spread out as two capsules in the morning and two capsules in the evening before meals.
Placebo: Four placebo capsules per day spread out as two capsules two times a day before meals. The placebo capsules contained isomalt (one of the excipients used in the kiwifruit composition) coloured with green food colouring to match the colour of the kiwifruit composition. All packaging was identical.
Patients: One hundred and forty subjects with constipation (and meeting all other selection criteria) were recruited into the clinical trial from over four hundred original volunteers, from this, during the trial there were six drop outs all due to personal reasons. This resulted in 67 subjects in the placebo group and 67 in the treatment group. The selection criteria was based on selecting normal healthy subjects who had recently undergone a change in bowel movement and had developed constipation defined as no more than three bowel movements per week. Long-term constipation subjects who had taken a range of treatments for constipation were excluded from the trial. The clinical assessment was carried out in accordance with the information required to complete the assessment form. The subjects were selected based on their medical history documentation and through an interview with a doctor.

### Trial Design and Grouping

The clinical trial was undertaken according to the standard protocol of human defecation test as stated in the “Technical Criterion for Examination and Evaluation of Health Food” issued by the Ministry of Health of the Peoples Republic of China in 2003. A double-blinded placebo based clinical trial was carried out. The trial was designed using both self-control and group-control models. Depending on the presence and intensity of the following constipation symptoms including: defecation frequency, defecation conditions, and faecal characteristic combined with consideration of the participant's age and gender, 134 subjects were selected and divided into 2 groups: 67 subjects in the test group and 67 subjects in the control (placebo) group.

Side Effects: AU subjects were trained to identify any possible side effects and were advised to contact a doctor in the trial immediately if any possible side effects were observed. Doctors also checked on a daily basis for side effects. Side effects that were of particular interest included; diarrhoea, abdominal pain, discomfort, bloating, flatulence, hydrogen sulphide or ammonia smelling faeces or other abnormal smells, and any rectal bleeding.

No side effects whatsoever were observed by any subject or by any doctor who examined them.

The patients were then started on the following regime:

Washout Period (weeks 1-2): Prior to the start of the feeding period (weeks 1-2), there was a fourteen day wash out period in which the subjects were prevented from using any dietary supplement or medicine for the treatment of constipation.
Treatment Period (14 days weeks 3-4): For the treatment phase (14 days—weeks 3-4) of the trial each of the 134 human subjects was given four capsules a day, either of the placebo or of the kiwifruit composition. Over this treatment period the number of bowel movements, constipation sensation as determined by the Rome II Criteria and faecal characteristics according to the Bristol Stool Chart were assessed.
Follow-up Period (weeks 5-6): For the follow-up period (weeks 5-6) of the trial the 134 subjects were not allowed to consume any product for constipation, with symptoms as described above continually monitored. Any subject suffering from chronic constipation was manually assisted by a doctor.

During this six week trial period, the subjects maintained their previous diet which had been assessed as a normal diet.

### Main Equipments and Reagents

### CX-4 Automatic Biochemistry Analyzer (Beckman Coulter, USA)

### JT-IR Tri-classified Blood Cell Analysis (Beckman Coulter, USA)

Uritest Urine analyzer (Medical Electronic Factory of Guilin City, China)

Total protein assay kit, Albumin assay kit, GPT assay kit, GOT assay kit, TG assay kit, TC assay kit, Blood uric acid assay kit, Creatinine assay kit, and Urea nitrogen assay kit, purchased at Beijing Biosino Biotechnology Company Ltd of China.

### Parameters Measured:

### Safety

Inclusive of mental disorders, any form of dieting, sleeping disturbance, diarrhoea, respiratory conditions and changes in blood pressure, etc.

### Blood, Urine, and Faecal Examination

Blood routine examination: Red blood cell count; white blood cell count, platelet, and hemoglobin, etc.

Urine routine examination: specific gravity, color, pH, protein, glucose, uric acid, bilirubin, urobilinogen, nitrite, white blood cell, and occult blood.

Faecal routine examination: appearance characteristics, red blood cell presence, white blood cell appearance, occult blood, presence of parasites and/or eggs.

### Liver and Kidney Function Examination

Total serum protein, albumin, GPT, GOT, blood glucose, creatinine, and urea nitrogen.

### Chest X-Ray, Electrocardiogram and B-Mode Sonography (Once Before Clinical Trial Started)

### Adverse Reactions

Nausea, flatulence, diarrhea, abdominal pain, and abnormal defecation were to be recorded if present.

Assessment and Compliance: All subjects had been initially assessed by a doctor to determine their degree of constipation. During the washout period all subjects were either visited by a doctor or they visited a doctor on a daily basis. Once the treatment phase of the trial started all subjects were contacted twice daily via a combination of phone calls and visits by/or to the doctors involved in the trial. During the follow-up period all subjects were either visited by a doctor, or they visited the doctor on a daily basis.

### Functional Parameters

During the six week trial period, doctors inquired and recorded daily the condition of each subject during the periods of: the 14 days wash out period, the 14 days treatment period, and the 14 days follow up period.

### Daily Faecal Times

Actual faecal times of each subject were recorded and statistically analyzed.

### Defecation Condition

The defection condition was categorized using the following classes. Class I to IV was measured by the defecation difficulty degree.
Class I (score 0): Normal defecation
Class II (score 1): Only a sense of tenesmus and discomfort
Class III (score 2): Obvious sense of tenesmus and discomfort, or urination was frequent while quantity was little and it was difficult to excrete; less abdominal pain or anal burning sensation
Class IV (score 3): Frequent abdominal pain, or anal burning sensation, impact to defecation

### Faecal Characteristics

The Bristol Stool Chart System was used to measure the faecal characteristics as detailed below. However, please note the numbering used below is that which is used when referring to constipation and varies from that of the traditional Bristol Stool Chart for overall faecal characteristics.

0 Class 0 (score 0): Snake or sausage like, smooth and soft; sausage-like, but there are cracks in the surface; soft conglomeration with clear edge (easy to be excreted)
1 Class I (score 1): Sausage-like, with conglomeration; loose, massive with rough edge, slurry-like stool.
2 Class II (score 2): Separated hard group, like stone (difficult to be excreted)
3 Class III (score 2): Separated hard group, like stone (difficult to be excreted)

### Data Analysis

SPSS 11.5 was used to analyze the mean and Standard Deviation of the above parameters obtained before and after the trial. Self-comparison data and group-comparison data was analyzed using paired-t-test and group-t-test, respectively. Sex proportionality was analyzed by χ² and exact probabilities.

### Criteria Set for Determining Clinical Significance for the Trial

### Estimate of Clinically Relevant Difference

Clinical significance in the trial is deemed to be “the statistically significant relief of constipation by at least one class as defined under “relief from constipation” in the protocol”

### End Point

The primary end point is defined as:

“The statistically significant (p<0.05) relief of constipation in comparison to the placebo”

This will be determined by the independent assessment of each subject across the experiment period against the placebo group.

A positive clinical trial result is deemed relevant if it meets the following criteria.

Self-comparison: defecation frequency is statistically significantly increased and the Score of either defecation condition or faecal characteristic was obviously reduced after the trial in comparison with those before trial.

Group-comparison: any of the defecation frequency, defecation condition, or faecal characteristics parameters showed statistically significantly improvement.

### Results

### Proportionality Comparison Between Test Group and Control Group

Total subjects were 134 (67 in each group), male 58, female 76, aged from 24 to 78 and were randomly assigned into either the placebo or active group.

Results (table 14) show that there is no statistical difference (P<0.05) of defecation frequency, defecation condition, faecal characteristic, age, and sex between the test and control groups prior to the clinical trial.

TABLE 14
Proportionality comparison between groups before trial

| | Test group | Control Group | |
|---|---|---|---|
| Parameters | (n = 67) | (n = 67) | P value |
| Defecation | 1.94 ± 0.32 | 1.94 ± 0.32 | 1.000 |
| frequency (times/week) | | | |
| Defecation | 1.35 ± 0.37 | 1.36 ± 0.32 | 0.857 |
| condition (score) | | | |
| Faecal | 1.55 ± 0.51 | 1.55 ± 0.58 | 0.977 |
| characters (score) | | | |
| Age | 51.34 ± 13.48 | 53.67 ± 14.34 | 0.335 |
| Sex (Male/Female) | 31/36 | 27/40 | 0.486 |

Values shown are +/−SD

### Safety Parameters

All of the safety parameters were within the normal range, and no significant change, whether self-compared or group-compared, was observed. These findings demonstrate the safety of the kiwifruit composition for human consumption.

### Functional Parameters

### Weekly Defecation Frequency

TABLE 15
Defecation frequency before and after trial

| | | Defecation frequency (Times/week) | | | | | | |
|---|---|---|---|---|---|---|---|---|
| | N | Before trial | During trial | P¹ | P² | After trial | P¹ | P² |
| Test | 67 | 1.94 ± 0.32 | 2.13 ± 0.40* | 0.003 | 0.697 | 2.46 ± 0.66*# | 0.000 | 0.001 |
| group | | | | | | | | |
| Control | 67 | 1.94 ± 0.32 | 2.10 ± 0.48 | | | 2.15 ± 0.36 | | |
| group | | | | | | | | |

P¹: self-compared* self-compared P < 0.05
P²: group-compared, # self-compared P < 0.05
Values shown are +/− SD

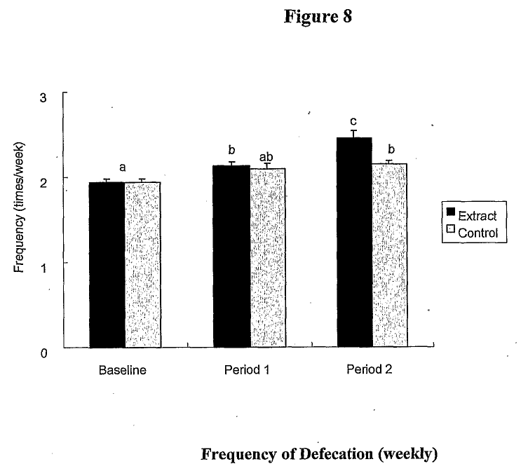

Results (table 15) show that the test group had a higher defecation frequency during and after the trial than that before the trial (P<0.05) when self compared, and also had a higher defecation frequency after the trial than that of the control group (P<0.05). However, as a group comparison this significant increase did not occur until the follow-up period (weeks 5-6). This is most likely due to the prebiotic activity rebalancing the gut micro-flora. Values shown are +/− standard deviation. These changes in the group comparison results can be seen in FIG. 8. FIG. 8 shows that at the end of the washout period (baseline) the frequency of defecation per week was 1.94. During the clinical trial feeding period (period 1) this increased in both the placebo and treatment groups. During the follow-up period, the frequency of defecation continued to rise in the treatment group and was significantly higher than in the placebo group. Standard error bars are shown.

### Defecation Condition

TABLE 16
Scores of the defecation condition before, during and after trial

| | | Scores of defecation condition | | | | | | |
|---|---|---|---|---|---|---|---|---|
| | N | Before trial | During trial | P¹ | P² | After trial | P¹ | P² |
| Test | 67 | 1.35 ± 0.37 | 1.06 ± 0.37* | 0.000 | 0.997 | 0.94 ± 0.31* | 0.000 | 0.378 |
| group | | | | | | | | |
| Control | 67 | 1.36 ± 0.32 | 1.06 ± 0.38 | | | 0.99 ± 0.34 | | |
| group | | | | | | | | |

P¹: self-compared* self-compared P < 0.05
P²: group-compared, # self-compared P < 0.05
Values shown are +/− SD

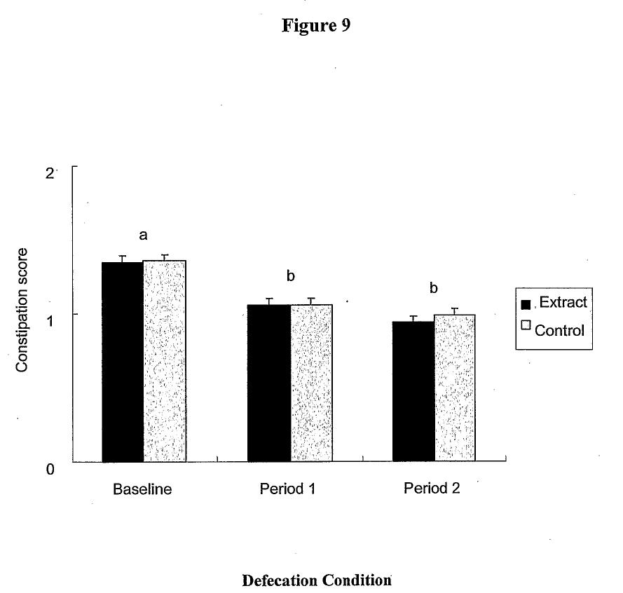

Results shown in table 16 and FIG. 9 demonstrate that both the test and placebo groups had a lower score of defecation condition during and after the trial than that before the trial (P<0.05). Values shown are +/− standard deviation. However, there is a significant difference both during and after the trial for the test group when self compared. FIG. 9 shows changes in the constipation score as measured according to the Rome II criteria. As can be seen both the treatment and placebo groups decreased in response to the trial. While this was not statistically significant there is an indication of ongoing benefit (active score decreasing faster than placebo) which indicates the need for either higher dosage or longer feeding period. Standard error bars are shown.

### Faecal Characteristic

TABLE 17
Scores of faecal characteristics before, during and after the trial

| | | Scores of faecal characteristics | | | | | | |
|---|---|---|---|---|---|---|---|---|
| | N | Before trial | During trial | P¹ | P² | After trial | P¹ | P² |
| Test | 67 | 1.55 ± 0.51 | 1.19 ± 0.53* | 0.000 | 0.118 | 1.19 ± 0.49*# | 0.000 | 0.003 |
| group | | | | | | | | |
| Control | 67 | 1.55 ± 0.58 | 1.34 ± 0.54 | | | 1.43 ± 0.40 | | |
| group | | | | | | | | |

P¹: self-compared* self-compared P < 0.05
P²: group-compared, # self-compared P < 0.05
Values shown are +/−SD

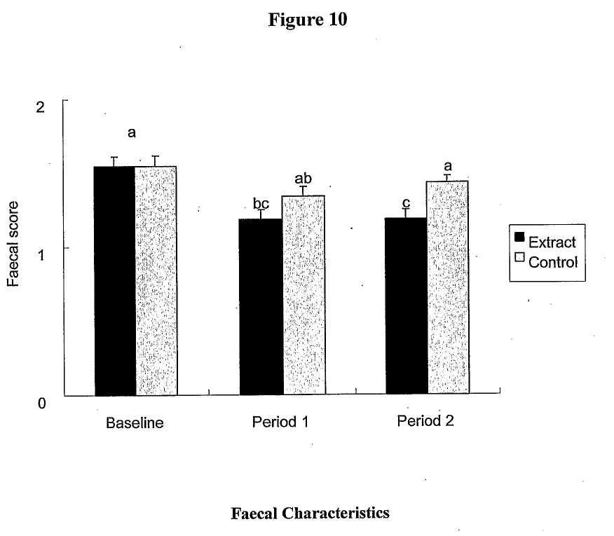

Results (table 17) show that the test group had a significantly lower score of faecal characteristic during and after the trial than that before the trial (P<0.05), and also had a lower score after the trial than that of the control group (P<0.05). Values shown are +/− standard deviation. FIG. 10 shows that the faecal score as determined by comparison to the Bristol Stool Chart in the group comparison shows statistical significance for the treatment group with a decrease in the faecal score (0 is normal). Standard error bars are shown.

### Conclusion

The results of the clinical trial demonstrate using self comparison that the patients who received the kiwifruit composition had a higher defecation frequency and a lower score of defecation condition combined with faecal characteristic during and after the trial than those before the trial (P<0.05), and also had a higher defecation frequency and a lower score of faecal characteristic after the trial than those of the control group (P<0.05). Using group comparison there was statistical significance for changes in defecation frequency and faecal characteristic in the follow-up period.

Self comparison is the analysis of changes in the individual subjects on a daily basis both in the test group and in the placebo group Thus statistical significance in the self compared group is based on the individual changes within the test group versus the self compared changes within the placebo group. Using this approach there was potent efficacy of the kiwifruit composition for constipation in comparison to the placebo. When using group comparison, statistical significance was achieved for defecation frequency and faecal characteristic during the follow up period. It is our premise that this significant change in the follow up period is most likely due to the prebiotic effect of the kiwifruit composition.

Analysis of the group comparison during and after the trial demonstrates, as seen in the animal trials, that the prebiotic effect takes time to build up in the system to relieve constipation. These findings strongly correlate with the animal trial results that a higher dose is required to sufficiently stimulate motility for a working digestive system as well as speed up the prebiotic effect and relieve constipation sensation including IBS symptoms.

### Second Clinical Trial

### Clinical Trial Protocol:

Patients: Seventy subjects with constipation were recruited into the clinical trial from three hundred original volunteers, from this there were 12 drop outs. During the trial, 10 dropped out due to personal reasons and 2 during the washout period due to spontaneous resolution of their constipation. This resulted in 30 subjects in the placebo group and 28 in the treatment group. The selection criteria was based on selecting normal healthy subjects who had recently undergone a change in bowel movement and had developed constipation defined as no more than 3 bowel movements a week. Long-term constipation subjects who had taken a range of treatments for constipation were excluded from the trial. The clinical assessment was carried out in accordance with the information required to complete the assessment form. The subjects were selected based on their medical history documentation and by an interview with a doctor. The age of the patients ranged from 23-65 years old.
Assessment and Compliance: All subjects had been initially assessed by a doctor to determine their degree of constipation. During the washout week all subjects were either visited by a doctor or they visited a doctor on a daily basis. Once the treatment phase of the trial started all subjects were contacted twice daily via a combination of phone calls and visits by/or to the doctors involved in the trial.
Side Effects: All subjects were trained to identify any possible side effects and instructed to contact a doctor in the trial immediately should any be experienced. Doctors also checked on a daily basis for side effects. Side effects that were of particular interest included; diarrhea, abdominal pain, discomfort, bloating, flatulence, hydrogen sulphide or ammonia smelling faeces or other abnormal smells, and any rectal bleeding.

No side effects whatsoever were observed by any subject or by any doctor who examined them.

Dosage: Six kiwifruit composition capsules per day spread out as two capsules three times a day before meals.
Placebo: Six placebo capsules per day spread out as two capsules three times a day before meals. The placebo capsules contained isomalt (one of the excipients as used in the kiwifruit composition) coloured with green food colouring to match the colour of the kiwifruit composition. All packaging was identical.

The patients were then started on the following regime:

Washout Period: (7 days—week 1) Prior to the start of the feeding period, there was a one week wash out period in which all trial subjects were prevented from using any dietary supplement or medicine for the treatment of constipation.
Treatment Period: (7 days—week 2) For the first week of the trial the fifty eight subjects were given six capsules a day, either of the placebo or of the kiwifruit composition. Over this treatment period the number of bowel movements, constipation sensation as determined by the Rome II Criteria and faecal characteristics were assessed.
Follow-up Period: (7 days—week 3) For the second week of the trial the fifty eight subjects were not allowed to consume any product for constipation, with symptoms as described above continually monitored.

### Results

### Change in Faecal Frequency

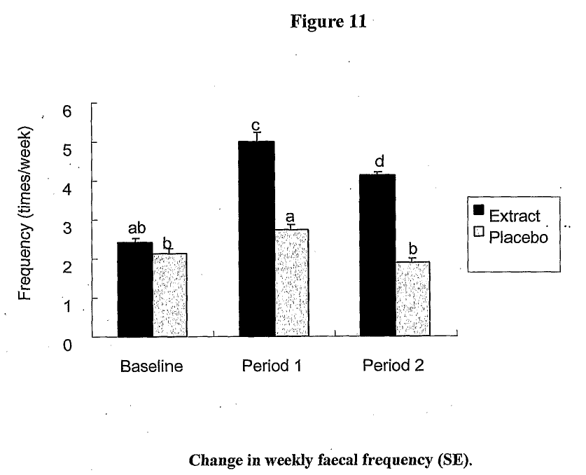

FIG. 11 shows the change in the number of bowel movements per week. The baseline represents the first week washout. Period 1 (7 days) represents the feeding period with either the kiwifruit composition at 6 capsules per day, or with 6 capsules of the placebo. Period 2 (7 days) represents the week following treatment in which no capsules were consumed by either group. There is a statistically significant difference (P<0.01) between the treatment group and the placebo group in both periods. In the first period this significant difference was calculated as P<0.0002. The error bars shown represent the Standard Error of the Mean. During the feeding period there is a small placebo effect, but this is gone by the follow up period.

### Faecal Score Based on Bristol Stool Chart

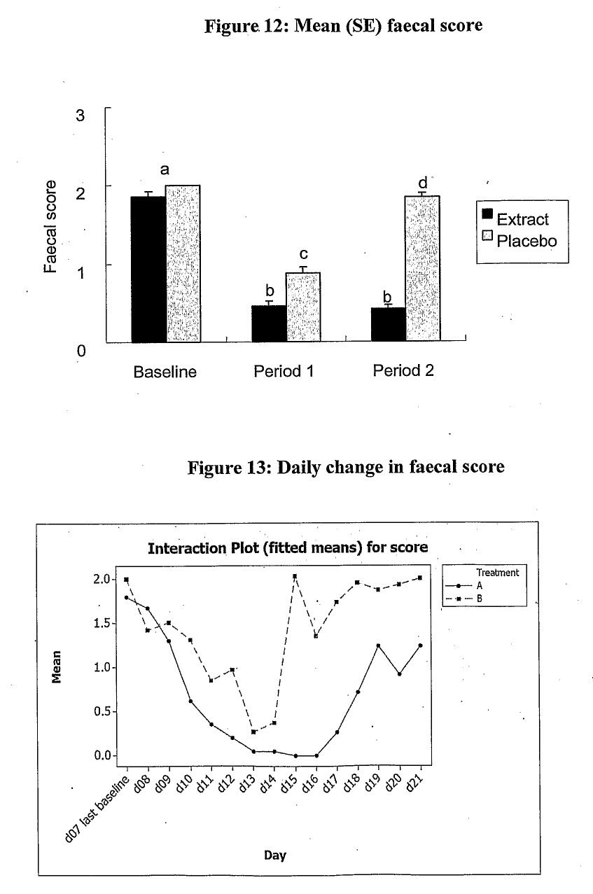

FIG. 12 represents mean faecal scores achieved within each of the three periods (wash-out, feeding, follow up), with the baseline represented by the washout period. A faecal score of 0 is the ideal, and as can be seen, consumption of six capsules per day of the kiwifruit composition returns the subjects to near normal faeces within the seven day feeding period, and is retained during the follow up period.

Baseline faecal score (day 7 of washout) was 1.9 with a standard error of the mean at 0.04. Period 1 represents the feeding period with either the kiwifruit composition (A) at 6 capsules per day, or with 6 capsules of the placebo (B). Period 2 represents the following week in which no capsules were consumed. The first week taking the capsules there is a potent placebo effect. There is statistical significance during the feeding period for the kiwifruit composition treatment for faecal score.

By the second week, when no capsules were being consumed the faecal score remained very low for the treatment group, indicating an apparent benefit from having taken the kiwifruit composition the week before. For the placebo group the score was increasing again and thus the stools were becoming harder again. There remains a statistically significant difference (P<0.01) between the treatment group and the placebo group during the follow up period.

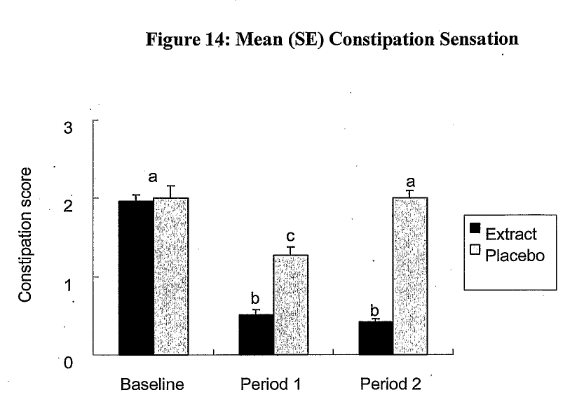

FIG. 13 represents the daily changes in the faecal score as determined by the Bristol Stool Chart criteria as described above. Note the reversal of the placebo effect at the end of the feeding period versus the continued benefit in the active group for 2 days before the score begins to return to the pre-feeding level. Although the mean scores for the feeding and follow up periods show excellent results, analysis of the daily results indicates that continued feeding of the kiwifruit composition has potential to achieve a mean score very close to zero. Note the consistency of the results with those for constipation sensation below.

### Changes in Constipation Sensation:

FIG. 14 shows the results for constipation sensation. It represents the weekly mean scores for constipation sensation, as determined by the Rome II criteria described above. To interpret FIG. 14, baseline represents the washout period, period 1 represents the seven day feeding period and period 2 the seven day follow up period. A constipation sensation score of 0 is normal, and as can be seen in the graph, consumption of six capsules per day of the kiwifruit composition returns the subjects to near normal sensation within the seven day feeding period, and is retained during the follow up period.

### Conclusion

The primary aim of these clinical trials was to determine whether the compositions of the present invention had statistically significant efficacy as a constipation relief product.

The anecdotal evidence provided at the start of the research suggested that two capsules per day was the recommended dose for relieving constipation. However, results from the animal trials strongly indicated that a higher dose was required for significance, hence the decision to undertake two trials at different dosage levels. Although significance was achieved at both 4 and 6 capsules per day, there was definitely a stronger effect at 6 capsules per day. Comparison of the findings between the animal and clinical trials shows a direct correlation in dosage response.

It is important to realize when examining the results for faecal characteristic and constipation sensation that two graphs are presented for each in the second clinical trial. One of these graphs shows the overall weekly change, whilst the other the daily changes. The daily changes highlight the potency of the placebo effect, but they also highlight the difficulty in analyzing daily changes in constipation. Since on any given day only a few subjects will be having a bowel movement, and thus the numbers shown per day are variable when it comes to frequency and stool characteristics. Nevertheless the trend in both cases indicates that continued use of the compositions of the present invention has the potential to achieve an ongoing result close to the ideal score.

Overall the results of the animal and clinical trials demonstrate to intense scientific scrutiny statistical significance for the relief of constipation. The clinical trials show statistically significant improvement in IBS symptoms (tenesmus, bloating and abdominal pain) according to Rome II criteria and in faecal characteristics according to the Bristol Stool Chart. These findings show that the compositions of the present invention have efficacy, not only for the relief of constipation, but also potentially for other digestive health benefits.

In summary, the major findings of these animal and clinical trials are:
1. The compositions of the present invention significantly improve constipation;
2. The compositions of the present invention are safe for human consumption;
3. There is a dose response correlation with efficacy;
4. There is a direct correlation between the animal and clinical trials in dose and efficacy;
5. There are no negative side effects including diarrhoea;
6. The results show during the follow up period there is still a beneficial effect from use of the compositions of the present invention;
7. The prebiotic effect takes approximately two weeks to establish in humans;
8. The enzyme in the compositions stimulates peristaltic motility;
9. The compositions of the present invention have a three way mode of action: the enzymes enhance gut motility, the prebiotic material improves healthy gut microflora, and the fibre assists in bulking the stool.

The results suggest that a person with particularly severe constipation should start on a higher dose and reduce the dose over time.

### Advantages

The pharmaceutical or nutritional compositions of the present invention provide a number of advantages, including but not limited to the following:
a) Promotion of healthy microflora in the gut by increasing beneficial bacteria and reducing undesirable microbes or pathogenic bacteria so that the ratio of beneficial/pathogenic bacteria in turn results in beneficial health effects for the host;
b) Improvements to long term bowel health;
c) Promotion of regular bowel movements;
d) Assists the body to absorb the optimal level of nutrients from food;
e) Allows the release of the effective concentrated material directly to the stomach where it can begin working immediately. In particular, the enzyme suppressors in the saliva are by-passed which allows the enzyme component of the composition to be released to the gut without any degradation. Administration in such a concentrated form is a lot more effective and easier than having to obtain the effective components of the composition from food and thus having to eat significant amounts of the appropriate food groups, such as raw fruit and vegetables.
f) The compositions of the present invention can be completely natural products derived from commonly consumed fruit such as kiwifruit, and as such there are very little, if any, risks of side effects or health threats to consumers except in the case of allergies to any of the particular ingredients.

### VARIATIONS

It will of course be realised that while the foregoing has been given by way of illustrative example of this invention, all such and other modifications and variations thereto as would be apparent to persons skilled in the art are deemed to fall within the broad scope and ambit of this invention as is hereinbefore described.

1-11. (canceled)

12. A method for the treatment or prevention of digestive dysfunction and/or gastrointestinal tract disorders by promoting the growth and/or activity of at least one type of beneficial bacteria present in the gut by administering an effective amount of a pharmaceutical or nutritional composition comprising a powdered kiwifruit extract comprising prebiotic material, enzymes and fibre derived from kiwifruit of the genus Actinidia.

13. The method of claim 12, wherein the beneficial bacteria are selected from the group consisting of probiotic bacteria, including bifidobacteria and lactobacilli.

14. The method of claim 13, wherein the probiotic bacteria are selected from the group consisting of Lactobacillus reuteri, Lactobacillus acidophilus, Pediococcus acidilactici, and Lactobacillus plantarum.

15. The method of claim 12, wherein the composition also inhibits or suppresses the growth of at least one type of pathogenic bacteria present in the gut.

16. The method of claim 15, wherein the pathogenic bacteria are selected from the group consisting of bacteroides, clostridia, coliforms, and sulphate reducing bacteria.

17. The method of claim 16, wherein the pathogenic bacteria are selected from the group consisting of Escherichia coli, Salmonella typhimurium, and Staphylococcus aureus.

18. The method of claim 12, wherein the total amount of prebiotic material present in the kiwifruit extract is in the range of about 10 to 35% w/w.

19. The method of claim 12, wherein the prebiotic material includes at least one compound selected from the group consisting of oligosaccharides, polysaccharides, disaccharides, monosaccharides, cellulose, chlorophyll, lignin and pectins.

20. The method of claim 19, wherein the prebiotic material includes at least one compound selected from the group consisting of fructo-oligosaccharides and gluco-oligosaccharides.

21. The method of claim 12, wherein the kiwifruit extract has a proteolytic enzyme activity in the range of 100-400 U/mg.

22. The method of claim 21, wherein the kiwifruit extract has a proteolytic enzyme activity in the range of 170-250 U/mg.

23. The method of claim 12, wherein the fibre in the kiwifruit extract is present in the range of about 5 to 15% w/w.

24. The method of claim 12, wherein the composition is in the form of tablets or capsules.

25. The method of claim 24, wherein each tablet or capsule contains about 535 mg of the kiwifruit extract.

26. The method of claim 24, wherein each tablet or capsule further comprises one or more excipients selected from the group comprising isomalt, magnesium stearate and silicon dioxide.

27. The method of claim 12, wherein the composition is in the form of a foodstuff or beverage.

28. The method of claim 13, wherein the total amount of prebiotic material present in the kiwifruit extract is in the range of about 10 to 35% w/w.

29. The method of claim 14, wherein the total amount of prebiotic material present in the kiwifruit extract is in the range of about 10 to 35% w/w.

30. The method of claim 15, wherein the total amount of prebiotic material present in the kiwifruit extract is in the range of about 10 to 35% w/w.

31. The method of claim 16, wherein the total amount of prebiotic material present in the kiwifruit extract is in the range of about 10 to 35% w/w.
